(19) United States
(12) Patent Application Publication
(10) Pub. No.: US 2015/0157648 A1
(43) Pub. Date: Jun. 11, 2015

(54) NUTRITIONAL APPROACH TO IMPROVING ATHLETIC PERFORMANCE AND REDUCING INJURY WITH L-ERGOTHIONEINE AND/OR VITAMIN D2

(71) Applicant: Entia Biosciences, Inc., Sherwood, OR (US)

(72) Inventor: Marvin S. Hausman, Stevenson, WA (US)

(21) Appl. No.: 14/410,754

(22) PCT Filed: Jun. 26, 2013
(86) PCT No.: PCT/US2013/047853

§ 371 Date: Dec. 23, 2014

Related U.S. Application Data
(60) Provisional application No. 61/664,418, filed on Jun. 26, 2012.

(51) Int. Cl. A61K 31/592; A61K 31/198; A61K 36/07; A61K 31/4164; A61K 35/748

(52) U.S. Cl. CPC A61K 31/592; A61K 31/4164; A61K 35/748; A61K 36/07; A61K 31/198

(57) ABSTRACT

Nutritional products, compositions, functional ingredients, medical foods, pharmaceutical preparations and methods of use are disclosed for support and improvement of athletic performance (strength, flexibility, endurance, balance, and conditioning), and prevention and repair of injuries, including improving or maintaining muscle mitochondrial health, stimulating muscle mitochondrial biogenesis as well as other indications as set forth herein. In addition, pharmaceutical, nutraceutical and nutritional medical food compositions that are useful in conditions, normal or abnormal, associated with mitochondrial dysfunction and altered biogenesis in organ systems consisting of skeletal muscle, smooth muscle and cardiac muscle are also disclosed. L-Ergothioneine (also referred to as Ergothioneine or ET), Vitamin D2 (Ergocalciferol) and/or other antioxidants are disclosed for use in pharmaceutical, nutraceutical and nutritional medical food compositions for uses to stimulate production of bioactive molecules, including but not limited to glutathione, alpha synuclein, and IL-6.

### FIELD OF THE INVENTION

This invention relates to the application of L-Ergothioneine and/or Vitamin D2 to physiologically support and improve athletic performance (strength, endurance, balance, and conditioning), and to prevent and repair injuries. This invention also relates to the maintenance of cellular mitochondrial health and stimulation of mitochondrial biogenesis, by the use of L-Ergothioneine (also referred to as Ergothioneine, Ergo, or ET), Vitamin D2 (Ergocalciferol) and/or other antioxidants. This invention also relates to pharmaceutical, nutraceutical, food ingredients, and nutritional medical food compositions to mobilize iron from natural and/or sequestered toxic pools, increase blood levels of iron and percentage iron saturation, stabilize iron in its normal 2⁺ charge for proper oxygen binding and carrying, stimulate red blood cell production with increased levels of hemoglobin, and stimulate production of bioactive molecules, including but not limited to glutathione, alpha synuclein, and IL6. It also relates to pharmaceutical, nutraceutical and nutritional medical food compositions that are useful in conditions, normal or abnormal, associated with mitochondrial dysfunction and altered biogenesis, as well as DNA damage and mutations. The extraction of Ergothioneine and Vitamin D2 from whole food sources and bacterium for use in nutritional products and treatments is also disclosed.

### BACKGROUND OF THE INVENTION

Early scientific studies relating to Ergothioneine identify its possible role as an antioxidant, through the incorporation of iron into the “heme” molecule (Goldberg, A. The Enzymatic Formation of Haem by the Incorporation of Iron into Protoporphyrin; Importance of Ascorbic Acid, Ergothioneine and Glutathione, Brit. J. Haemat. (1959), 78:150-153). It was suggested that Ergothioneine plays a part in the maintenance of hemoglobin iron in the reduced state. This potent antioxidant also appears to have a role in maintaining the function of erythrocytes and protecting them from oxidative damage (Touster, O. Estimation of Blood Ergothioneine by Determination of Bromine-Labile Sulfur. J. Biol. Chem. (1951) 188: 371-377 Chapman, P. K. Biomed. Biochem. Acta., 1143-1149 (1983); Chapman, P. K., The antioxidant action of L-Ergothioneine Biomed. Biochem. Acta. (1983), 42:1143-1149)). The ability of Ergothioneine to protect hemoproteins such as hemoglobin within erythrocytes against oxidation probably could explain the millimolar concentrations seen in these cells (Arduini, A., Possible mechanism of inhibition of nitrite-induced oxidation of oxyhemoglobin by ergothioneine and uric acid. Arch Biochem Biophys. (1992) 294(2):398-402; Spicer, S. W. et al, Ergothioneine depletion in rabbit erythrocytes and its effect on methemoglobin formation and reversion. Proc. Soc. Exp. Biol. & Med. (1951), 77:418-420). The avidity by which dietary Ergothioneine is incorporated into tissues, the tenacity with which it is retained, and its unique non-uniform pattern of tissue distribution support the physiological importance of this molecule.

A unique Ergothioneine Transporter (ETT) has been identified in human cells with the gene, SLC22A4, coding for an integral membrane protein, OCTN1, and the key substrate of this transporter is L-Ergothioneine (ET) (Gründemann, D. H., Discovery of the Ergothioneine Transporter. Proc. Natl. Acad. Science (PNAS) (2005), 102(14), 5256-5261). The ETT is described in further detail in PCT/EP2005/005613 and U.S. patent application Ser. No. 11/569,451, titled “Identification of Ergothioneine Transporter and Therapeutic Uses Thereof,” such references are incorporated herein by reference in its entirety. ETT was identified as the first molecular marker of Ergothioneine activity proving to be necessary for the supply of ET primarily to erythrocyte progenitor cells and to monocytes. Using real-time PCR, strong expression of ETT in bone marrow was found (Kobayashi D. et al., Expression of organic cation transporter OCTN1 in hematopoietic cells during erythroid differentiation. Exp. Hematol. (2004), 32:1156-62), suggesting that ETT charges developing erythrocytes with available ET, protecting erythrocytes against damage related to HbFeIV-O (ferryl hemoglobin). HbFeIV-O species is a highly reactive intermediate in the autocatalytic oxidation, caused by many xenobiotics, of HbFeIIO2 to methemoglobin (HbFeIII) and is also considered a starting point for detrimental radical reactions including heme degradation (Alayash, A., Oxygen therapeutics: Can we tame haemoglobin? Nat Rev Drug Discov (2004) 3: 152-159).

Further data on the important role of Ergothioneine as a natural cytoprotectant is established. (Paul, B. and Snyder, S., The Unusual Amino Acid L-Ergothioneine is a Physiologic Cytoprotectant. Cell Death & Differentiation (2010), 17:1134-1140). Using RNA interference, cells were depleted of its transporter and cells lacking ETT were more susceptible to oxidative stress, resulting in mitochondrial DNA damage, protein oxidation and lipid peroxidation. ET was found to be as potent as glutathione, leading to the discovery that Ergothioneine may represent a new vitamin whose physiologic roles include antioxidant cytoprotection and potential protection against radiation disease, associated DNA mutations and development of cancer.

The potency of Ergothioneine as an electron donor and protector of DNA damage is further revealed by its ability to prevent copper-induced oxidative damage to DNA and proteins. It is stated: “We conclude that ergothioneine is a potent, natural sulfur-containing antioxidant that prevents copper-dependent oxidative damage to biological macromolecules by forming a redox-inactive ergothioneine-copper complex (Zhu, B. Z. et al, Ergothioneine prevents copper-induced oxidative damage to DNA and protein by forming a redox-inactive ergothioneine-copper complex, Chem Res Toxicol. (2011) 24(1):30-4).

Ergothioneine is a unique, naturally occurring antioxidant that is found in most plants and animals, but highly concentrated in mushrooms. The accumulation, tissue distribution and scavenging properties, all highlight the potential for Ergothioneine to function as a physiological antioxidant. An excellent article by Cheah and Halliwell reviews the current state of knowledge on Ergothioneine; this article is part of a Special Issue entitled: Antioxidants and Antioxidant Treatment in Disease (Cheah, I. K., and Halliwell, B. Ergothioneine; antioxidant potential, physiological function and role in disease. Biochimica et Biophysica Acta 1822: 784-793). It has been established that Ergothioneine cannot be synthesized by humans and therefore is available only from dietary sources, which was confirmed in human bioavailability studies conducted in the Department of Food Science, Pennsylvania State University. (Weigand-Heller et al. The bioavailability of ergothioneine from mushrooms (Agaricus bisporus) and the acute effects on antioxidant capacity and biomarkers of inflammation. Preventive Medicine (2012), 54:575-578). A postprandial time course study of varying mushroom doses (0 g, 8 g, and 16 g) was used to evaluate the bioavailability of L-Ergothioneine (ET) from mushrooms in healthy men, using a randomized, cross-over, dose-response, postprandial time-course design. ET was administered through a mushroom test meal containing 8 g and 16 g of mushroom powder, equivalent to about 1 or 2 servings of fresh mushrooms respectively. Postprandial red blood cell concentrations of ET were measured. Plasma glucose, triglycerides, HDL, LDL and total cholesterol also were monitored. Biomarkers of inflammation and oxidative stress were evaluated using C-reactive protein and ORACₜₒₜₐₗ. According to the results, ET was bioavailable and a trend in the postprandial triglyceride response indicated that there was a blunting effect after both the 8 g and 16 g ET doses compared with the 0 g dose. Despite ET's antioxidant properties, ORACₜₒₜₐₗ values decreased after the 8 g and 16 g mushroom meal. The investigators stated that ET exerts antioxidant properties through multiple mechanisms aside from scavenging free radicals and that due to the various mechanisms of action, antioxidant capacity would be better measured by an oxidative stress biomarker.

This study convincingly indicated that L-Ergothioneine is bioavailable in humans through the consumption of mushrooms (peak of ET appeared in red blood cells (RBC) after only 2 hours of mushroom consumption), providing further supportive evidence for the ETT active transporter. The appearance of ET within red blood cells in such a short time after ingestion of mushrooms strongly suggests that human tissues and cells contain an active mechanism of transport for ET

These and other valuable health benefits of ET-enhanced mushrooms are disclosed in U.S. patent application Ser. Nos. 12/887,276 and 12/386,810, titled “Vitamin D2 Enriched Mushrooms and Fungi for Treatment of Oxidative Stress, Alzheimer's Disease and Associated Disease States,” and “Methods and Compositions for Improving the Nutritional Content of Mushrooms and Fungi,” respectively, which are herein incorporated by reference in its entirety. Mushrooms are a valuable health food—low in calories, high in vegetable proteins, chitin, iron, zinc, fiber, essential amino acids, vitamins and minerals. They are also an excellent source of organic selenium compounds, riboflavin, pantothenic acid, copper, niacin, potassium and phosphorous. Selenium is needed for the proper function of the antioxidant system, which works to reduce the levels of damaging free radicals in the body. Selenium is a necessary cofactor of one of the body's most important internally produced antioxidants, glutathione peroxidase, and also works with vitamin E in numerous vital antioxidant systems throughout the body. Mushrooms are also a primary source of natural Vitamin D, in the form of D2, which is naturally present in very few foods. Most other natural food sources of Vitamin D, in the form Vitamin D3, are of animal, poultry or seafood origin.

Additional valuable benefits of Ergothioneine to health as well as food and beverage products are disclosed in PCT Application US08/56234 filed Mar. 7, 2008, U.S. patent application Ser. No. 12/529,859 and European Patent Application, Serial No. 08731684.0, along with Issued Canadian Patent No. 2,680,223, titled “Use of Ergothioneine as a Preservative in Foods and Beverages.”

Vitamin D is a fat-soluble vitamin that is naturally present in very few foods, added to others, and available as a dietary supplement. Vitamin D comes in two forms (D2 (ergocalciferol) and D3 (cholecalciferol)) which differ chemically in their side chains. These structural differences alter their binding to the carrier protein Vitamin D binding protein (DBP) and their metabolism, but in general the biologic activity of their active metabolites is comparable. It is also produced endogenously when ultraviolet rays from sunlight strike the skin and trigger Vitamin D synthesis. So one must either ingest Vitamin D or be exposed to UV radiation in the form of UV light, so that it may be synthesized endogenously. Most of the population is deficient in Vitamin D. The risks of sun exposure continue to gain attention, including the association of sun exposure with pre-cancerous (actinic keratosis) and cancerous (basal cell carcinoma, squamous cell carcinoma and melanoma) skin lesions—caused by loss of the skin's immune function, fine and coarse wrinkling of the skin, freckles, discoloration of the skin, and Elastosis (the destruction of the elastic tissue causing lines and wrinkles) is well documented. Thus, as people become more sensitive to the dangers of UV exposure, other dietary sources of Vitamin D become increasingly important for maintaining health.

There are two basic types of Vitamin D. Ergosterol is the basic building block of Vitamin D in plants and fungi. Cholesterol is the basic building block of Vitamin D in humans. When ultraviolet light from the sun hits the leaf of a plant or fungal tissue, ergosterol is converted into ergocalciferol, or Vitamin D2. In just the same way, when ultraviolet light hits the cells of our skin, one form of cholesterol found in our skin cells-called 7-dehydrocholesterol can be converted into cholecalciferol, a form of Vitamin D3. The liver and other tissues metabolize Vitamin D, whether from the skin or oral ingestion, to 25OHD, the principal circulating form of Vitamin D, by the enzyme CYP27B 1, the 25OHD-1αhydroxylase. 25OHD is then further metabolized to 1,25(OH)2D principally in the kidney, although other tissues such as epidermal keratinocytes and macrophages contain this enzymatic activity. 1,25(OH)2D is the principal hormonal form of Vitamin D, responsible for most of its biologic actions.

Vitamin D has many roles in human health, including modulation of neuromuscular and immune function, reduction of inflammation, maintaining blood levels of phosphorus and calcium, promotion of bone mineralization and calcium absorption, maintaining a healthy immune system, and regulating cell differentiation and growth. Recent studies have also shown a link between vitamin D deficiency and diseases such as cancer, chronic heart disease, inflammatory bowel disease and even mental illness. In addition, many genes encoding proteins that regulate cell proliferation, differentiation, and apoptosis are modulated in part by Vitamin D. Many laboratory-cultured human cells have Vitamin D receptors and some convert 25(OH)D to 1,25(OH)₂D. It remains to be determined what cells, tissues, and organs in the human body contain either D2, D3, or both vitamin receptors and what additional cells with Vitamin D receptors in the intact human can carry out this conversion from 25(OH)D to 1,25(OH)₂D.

The detrimental effects of inflammatory conditions involve interactive processes involving inflammation, free radicals, reactive oxygen species (ROS) and oxidative stress. Free radicals (or ROS) are unstable, short lived and highly reactive and are biologic markers of various inflammatory conditions, including for example, cytokines such as IL-2, TNF-alpha, nitric oxide, hydrogen peroxide and heat shock protein as well as activated peptides and biomarkers, such as amyloid precursor protein, beta-amyloid, and alpha synuclein, in neurodegenerative conditions such as Alzheimer's disease, Parkinson's disease, traumatic brain injury and post-traumatic stress disorder (PTSD). The effects of inflammatory processes and tissue damage caused by oxidative stress, free radicals and inflammatory processes relating to neuroinflammatory conditions are disclosed in U.S. patent application Ser. Nos. 12/887,276 and 13/363,579, titled “Anti-Inflammatory Approach to Prevention and Suppression of Post-Traumatic Stress Disorder, Traumatic Brain Injury, Depression and Associated Disease States,” which are herein incorporated by reference in their entirety.

Normal cellular and mitochondrial metabolism result in the production of free radicals. Free radicals are very unstable and react quickly with other compounds, trying to capture the needed electron to gain stability. Generally, free radicals attack the nearest stable molecule, “stealing” its electron. When the “attacked” molecule loses its electron, it becomes a free radical itself, beginning a chain reaction. Once the process is started, it can cascade, finally resulting in the disruption of a living cell. Antioxidants neutralize free radicals by donating one of their own electrons, ending the electron-“stealing” reaction. Ergothioneine is a potent antioxidant nutrient that has the capacity to donate an electron without becoming a free radical because it is stable in either form. Ergothioneine acts as a free radical scavenger and prevents cell and tissue damage.

Mitochondria are rod-shaped structures that are present in both plant and animal cells. The most important function of the mitochondria is to produce energy. The food that we eat is broken into simpler molecules like carbohydrates, fats, etc., in our bodies. These are sent to the mitochondrion where they are further processed to produce charged molecules that combine with oxygen and produce ATP molecules. This entire process is known as oxidative phosphorylation. In addition to their well-known function of supplying energy to a cell, mitochondria and their components participate in a number of other cellular activities. For example, mitochondria also control thermogenesis and the apoptosis process and are thus involved in the ageing process.

Mitochondrial biogenesis refers to processes of growth, amplification and healthy maintenance of the mitochondria. Mitochondrial biogenesis is a complex process involving both nuclear and mitochondrial players. “Mitochondrial biogenesis” as referred to in this patent includes all processes involved in maintenance and growth of the mitochondria, including those required for mitochondrial division and segregation during the cell cycle. Biogenesis within mitochondria results in increased oxygen usage and increased energy while engaged in exercise. Oxidative metabolism increases with greater concentrations of mitochondria and also results in increased endurance. “Skeletal muscle mitochondria are implicated with age-related loss of function and insulin resistance.” (Menshikova, E. V. et al, Effects of Exercise on Mitochondrial Content and Function in Aging Human Skeletal Muscle, J Gerontol. A Biol Sci Med Sci. (2006) 61(6): 534-540). “In conclusion, exercise enhances mitochondria electron transport chain activity in older human skeletal muscle, particularly in sub-sarcolemmal mitochondria, which is likely related to the concomitant increases in mitochondria biogenesis. The medicinal properties and usage of phytonutrients (or phytochemicals) in combination with Ergothioneine and vitamin D have utility for treating such inflammatory conditions and associated insulin resistance. Further evidence of the role of Vitamin D2 by itself or in combination with Ergothioneine and/or natural cofactors in mushrooms in combating oxidative stress was shown in a study entitled “UV Enriched Mushrooms with Elevated Levels of Natural Vitamin D2 Dramatically Improve Survival of Drosophila under Oxidative Stress Outperforms synthetic Vitamin D2 and D3 supplements, which showed no effect.” (Beelman et al, 2013; to be submitted for publication).

Ergogenic aids are substances, devices or practices that enhance an individual's energy use, production, or recovery. Nutritional supplements are a common recognized form of ergogenic aids. The use of nutritional substances to increase performance has become very widespread. An example includes supplementation with antioxidant vitamins to prevent muscle tissue damage associated with generation of oxygen free radicals during high-intensity exercise. “Recent reviews suggest that research regarding the value of antioxidant therapy for athletes is ambivalent. Some reviewers (Goldfarb, A., Antioxidants: Role of supplementation to prevent exercise-induced oxidative stress. Medicine and Science in Sports and Exercise (1993), 25: 232-236; Kanter, M., Free radicals and exercise: Effects of nutritional antioxidant supplementation. Exercise and Sport Sciences Reviews (1995), 23: 375-398)) note that further investigations are needed to determine the viability of antioxidant supplements in preventing exercise-induced lipid peroxidation and muscle damage. Conversely, other reviewers (Dekkers J., et al., The role of antioxidant vitamins and enzymes in the prevention of exercise-induced muscle damage. Sports Medicine 1996), 21: 213-238; Packer, L. Oxidants, antioxidant nutrients and the athlete. Journal of Sports Sciences (1997), 15: 353-363)) indicate substantial research suggests that dietary supplementation with antioxidant vitamins has favorable effects on lipid peroxidation and exercise-induced muscle damage. All reviewers indicate more research is needed to address this issue and to provide guidelines for recommendations to athletes” (Department of Health & Human Services, Nutritional Erogogenics & Sports Performance.) The potential application of Vitamin D to muscle function and athletic performance was recently shown by data from an orthopedic study that maintaining Vitamin D levels—as measured by 25(OH)D levels—could help diminish muscular weakness after intense exercise (Barker, T., Henriksen, V. T., Martins, T. B. et al. Higher serum 25-hydroxyvitamin D concentrations associate with a faster recovery time of skeletal muscle strength after muscular injury. Nutrients (2013), 5: 1253-1275).

Iron plays a critical role in cellular function and is both an essential nutrient and a potential toxicant to cells; as such, it requires a highly sophisticated and complex set of regulatory approaches to meet the demands of cells as well as prevent excess accumulation (Beard, J L., Iron biology in immune function, muscle metabolism and neuronal functioning. J Nutr. (2001), 131:568S-579S). Functional pools of iron change at various stages of iron status and the homeostasis of iron is essential for normal biochemical cellular functions in immune system function, nerve function, muscle function, and energy metabolism. Large gaps remain in the biological dose-response relationship between the severity of anemia and functional outcomes associated with disease development and treatment. Studies have examined the relationship between iron deficiency and hair loss. Dermatologists at the Cleveland Clinic Foundation believe that treatment for hair loss is enhanced when iron deficiency, with or without anemia, is treated (Trost, L. B., Bergfeld, W. F. and Calogeras, F., The diagnosis and treatment of iron deficiency and its potential relationship to hair loss. J. Am. Acad. Dermatol. 2006) 54(5): 824-44).

Another example cited: “Coenzyme Q10 (Ubiquinone)-Coenzyme Q10 (CoQ10), also known as ubiquinone, is a lipid with characteristics common to a vitamin. CoQ10 is found in the mitochondria in all tissues, particularly the heart and skeletal muscles. CoQ10 is also an antioxidant. CoQ10 supplementation has been used therapeutically for the treatment of cardiovascular disease because it may improve oxygen uptake in the mitochondria of the heart. Theoretically, improved oxygen usage in the heart and skeletal muscles could improve aerobic endurance performance.” (Melvin H. Williams, PhD, Eminent Scholar Emeritus Department of Exercise Science, Physical Education and Recreation Old Dominion University, Norfolk, Va. 23529-0196, Department of Health & Human Services, Nutritional Erogogenics & Sports Performance).

There are several well-known peptides and/or proteins that play key roles as antioxidants and inflammatory signaling molecules, including but not limited to glutathione and interleukin 6 (IL6).

IL-6 is an interleukin that acts as both a pro-inflammatory and anti-inflammatory cytokine. It is secreted by T cells and macrophages to stimulate immune response, e.g. during infection and after trauma, especially burns or other tissue damage leading to inflammation. In terms of host response to a foreign pathogen during infection, IL-6 has been shown, in mice, to be required for resistance against the bacterium Streptococcus pneumonia (van der Poll T, Keogh C V, Guirao X, Buurman W A, Kopf M, Lowry S F (1997), Interleukin-6 gene-deficient mice show impaired defense against pneumococcal pneumonia. J Infect Dis (1997) 176 (2): 439-444). IL-6 is also a “myokine,” a cytokine produced from muscle, and is elevated in response to muscle contraction (Febbraio M A, Pedersen B K., Contraction-induced myokine production and release: is skeletal muscle an endocrine organ? Exerc Sport Sci Rev. 2005) 33 (3): 114-119). It is significantly elevated with exercise, and precedes the appearance of other cytokines in the circulation. During exercise, it is thought to act in a hormone-like manner to mobilize extracellular substrates and/or augment substrate delivery (Petersen A M, Pedersen B K., The anti-inflammatory effect of exercise. J. Appl. Physiol. (2005), 98 (4): 1154-1162). Additionally, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10.

Glutathione has multiple functions: (1) It is the major endogenous antioxidant produced by the cells, participating directly in the neutralization of free radicals and reactive oxygen compounds, as well as maintaining exogenous antioxidants such as vitamins C and E in their reduced (active) forms; (2) regulation of the nitric oxide cycle, which is critical for life but can be problematic if unregulated; (3) it is used in metabolic and biochemical reactions such as DNA synthesis and repair, protein synthesis, prostaglandin synthesis, amino acid transport, and enzyme activation. Thus, every system in the body can be affected by the state of the glutathione system, especially the immune system, the nervous system, the gastrointestinal system and the lungs; and (4) it has a vital function in iron metabolism. Yeast cells depleted of or containing toxic levels of GSH show an intense iron starvation-like response and impairment of the activity of extra-mitochondrial ISC enzymes, followed by death (Chitranshu Kumar et al. Glutathione revisited: a vital function in iron metabolism and ancillary role in thiol-redox control. The EMBO Journal (2011) 30, 2044-2056).

Glutathione activity, muscle function, and endurance training was studied by M. Tonkonogi and colleagues. They concluded, “after endurance training: (1) the relative increase in maximal muscle fibre respiration exceeds that of whole-body oxygen uptake; (2) the sensitivity of mitochondrial respiration to ADP decreases; and (3) the impairment of oxidative function in skinned muscle fibres by ROS remains unchanged. (Tonkonogi, M., Walsh, B., Svensson, M., Sahlin, K. Effect of endurance training on oxidative and antioxidative function in human permeabilized muscle fibres. The Journal of Physiology (2000), 442: 379-388).

The role of glutathione in muscle function physiology is illustrated by the following statements: “Protein glutathiolation is an important post-translational protein modification that is increased under conditions of oxidative and nitrosative stress. It regulates the function of a variety of enzymes and is implicated in redox signaling, metabolic function, and apoptosis.” Taken as a whole, glutathiolation appears to be an important modification regulating bioenergetic function and cell death. Interventions that modulate protein thiolation levels in specific sub-domains could therefore have therapeutic potential in pathological conditions characterized by increased oxidative or nitrosative stress.” (Hill, B. G. et al, Regulation of Vascular Smooth Muscle Cell Bioenginergetic Function by Protein Glutathiolatoin, Biochim Biophys Acta. (2010); 1797(2): 285-295). Additional valuable health benefits of ET-enhanced mushrooms are disclosed in U.S. patent application Ser. No. 16/638,162, titled “Application of The Ergothioneine Transporter SLC22A4 and/or L-Ergothioneine to Targeted Diagnostic Identification and Treatment of Autoimmune Diseases,” which is herein incorporated by reference in its entirety. These autoimmune diseases include but are not limited to atherosclerosis, neurodegenerative diseases, diabetes mellitus, rheumatoid arthritis, and Crohn's disease.

Alpha-synuclein is a 140 amino-acid protein that is highly abundant in the brain, where it is predominantly expressed in the neocortex, hippocampus, substantia nigra, thalamus, and cerebellum. It is predominantly a neuronal protein, but can also be found in the neuroglial cells. Alpha-synuclein is also present in other tissues, including red blood cells. Point mutations in the SNCA gene, encoding for alpha-synuclein, and multiplications of the SNCA locus have been identified in families with autosomal-dominant forms of Parkinson's disease (PD). In PD, alpha-synuclein is found as a major component of Lewy bodies and Lewy neuritis. Alpha-synuclein also accumulates in dementia with Lewy bodies (DLB) and MSA. In MSA, alpha-synuclein is found predominantly within oligodendrocytes as cytoplasmic inclusions. These disorders share the accumulation of alpha-synuclein aggregates as a pathological feature and are collectively known as synucleinopathies. Additionally, alpha-synuclein was also identified as a component of amyloid from brain tissues of Alzheimer's disease (AD) patients.

It is an object of the present invention to provide a cost effective, natural whole food method to physiologically support and improve athletic performance (strength, flexibility, endurance, balance, and conditioning), and to prevent and repair injuries.

It is a further object of the invention to stimulate mitochondrial regeneration, maintain mitochondrial function and biogenesis within cells and tissues.

It is a further object of the invention to provide a composition, such as Ergo-D2, a potent anti-oxidant, anti-inflammatory nutritional product to improve muscle function, strength, balance, coordination and endurance as well as mitochondrial function and biogenesis within muscular cells and tissues.

It is a further object of the invention to provide a composition, such as ErgoD2™, a potent anti-oxidant, anti-inflammatory nutritional product, to increase blood levels of iron, increase percentage total iron saturation, increase numbers and quality of red blood cells, mean corpuscular hemoglobin concentration (MCHC), with resultant improved oxygen carrying capacity to cells and tissues.

It is a still further object of the invention to provide a composition, such as ErgoD2™ to stimulate production of signaling proteins and antioxidants involved in regeneration of cells and tissues, such as glutathione, IL6 and alpha synuclein. It is a further object of the invention to provide a composition, such as ErgoD2™, a potent anti-oxidant, anti-inflammatory nutritional product, to protect cells and tissues against radiation disease, DNA mutations and cancer.

It is a further object of the invention to provide a composition, such as ErgoD2™, a potent anti-oxidant, anti-inflammatory nutritional product, that has inherent antimicrobial, antioxidant food preservation actions, plus containing natural food taste enhancer (Umami) components.

It is a further object of the invention to reduce inflammatory reactions within muscles and joints with resultant improvement of range of motion, flexibility and reduction of pain.

A further object of the present invention is to provide a dietary supplement or other food or beverage products which are high in nutritional values, particularly Vitamin D2 and Ergothioneine that is extracted from natural whole food sources (including mushrooms, e.g. ErgoD2™) and/or bacterial sources.

It is another object of the invention to provide dietary supplements, dietary ingredients or other food or beverage products obtained from whole, natural sources (such as Spirulina or oats) to physiologically support and improve athletic performance (strength, flexibility, endurance, balance, and conditioning) and to prevent and repair injuries, as well as support mitochondrial function and biogenesis within cells and tissues.

It is an object of the present invention to diagnose the presence, absence, as well as varying concentrations of the Ergothioneine Transporter SLC22A4 within the membranes of cells and/or mitochondria in various tissues and organs with functional disorders, physiologic or pathogenic.

It is an object of the present invention to diagnose the presence, absence, as well as varying concentrations of the Ergothioneine Transporter SLC22A4 within the membranes of cells and/or mitochondria in disorders and conditions associated with autoimmune diseases (e.g. diabetes mellitus, rheumatoid arthritis, Crohn's disease, alopecia areata, psoriasis), anemia and including intestinal muscular dysfunction as an early hallmark of neurodegenerative diseases such as Parkinson's disease.

It is an object of the present invention to diagnose the presence, absence, as well as varying concentrations of the Ergothioneine Transporter SLC22A4 within the membranes of cells and/or mitochondria in neuromuscular disorders and conditions associated with autoimmune diseases including but not limited to Multiple sclerosis, Guillan-Barre syndrome which affect nerve function and produce muscle weakness.

These and other objects of the present invention will become apparent from the description of the invention which follows.

### SUMMARY OF THE INVENTION

Support and improvement of athletic performance (including for example, strength, flexibility, endurance, balance, and conditioning), and prevention and repair of injuries, including mitochondrial function within cells and tissues are provided according to the invention.

According to an embodiment, the invention creates an improved food or supplement product with a naturally enriched Vitamin D and Ergothioneine nutritional profile. According to an embodiment, the invention creates an improved food or supplement product with Ergothioneine, and optionally including Vitamin D and/or other antioxidants. The products according to the invention may be obtained from a variety of whole natural sources, including mushrooms, yeast, oats or barley or cyanobacteria, including Spirulina. The Ergothioneine may be combined with phytonutrients, Vitamin D enriched mushroom substrates (namely a mushroom or other fungi having enhanced content of Vitamin D or its analogs or derivatives), Beta glucans, chitin-glucans and/or other antioxidants such as turmeric and/or n-acetyl cysteine (NAC).

In an embodiment, the combination of Ergothioneine and Vitamin D stimulates production of antioxidant protein signaling molecules involved in cellular and tissue regeneration and biogenesis and mitochondrial regeneration. The compositions according to the invention may be provided as a daily supplementation regimen for prevention and/or as treatment regimens. In a further embodiment of the invention, the supplements or food product prevents, reduces and/or suppresses inflammation, oxidative stress and damage to cells and tissues, processes that occur during normal cellular and tissue metabolism and energy production, neutralizes damaging free radicals/cytokines, and induces production of protective antioxidants, such as glutathione and IL6.

It is a still further embodiment, that the combination of Ergothioneine and Vitamin D, such as ErgoD2™ (see Table 3), has the ability to inhibit the inflammation/insulin resistance axis within cells and tissues while at the same time preserving essential innate immune functions, resulting in better physiologic response to production of natural insulin.

It is still a further embodiment, that the combination of Ergothioneine and Vitamin D, such as ErgoD2™, has the ability to mobilize iron within various functional pools with resultant increase in blood iron levels and total percentage iron saturation resulting in better oxygen carrying capacity, quicker exercise muscle recovery time, and improved muscle contraction and strength.

In a further embodiment, the invention includes pharmaceutical compositions for support and improvement of athletic performance (strength, flexibility, endurance, balance, and conditioning), and prevention and repair of injuries, including mitochondrial function within cells and tissues

### DETAILED DESCRIPTION OF THE FIGURES

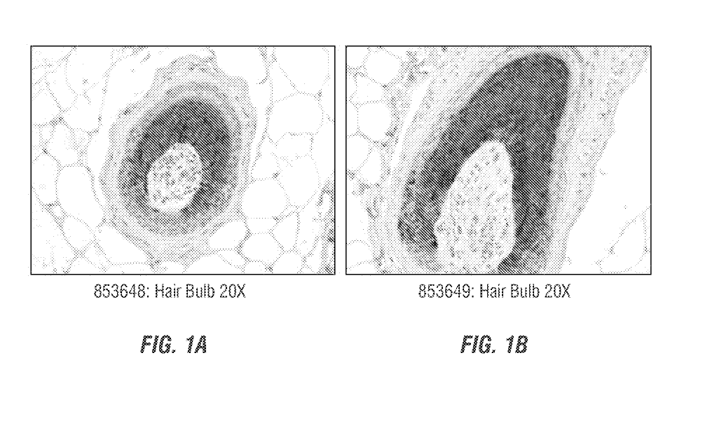

FIG. 1 (A-B) shows immunohistochemistry of hair follicles in normal scalp skin illustrating a marked increase in ETT within the stem cells of the hair bulb. Sample of scalp was obtained from a 17-year-old female who died of trauma. Hair follicles showed blush staining of the inner root sheath, with strong staining of the matrix keratinocytes of the hair root and faint staining of papilla.

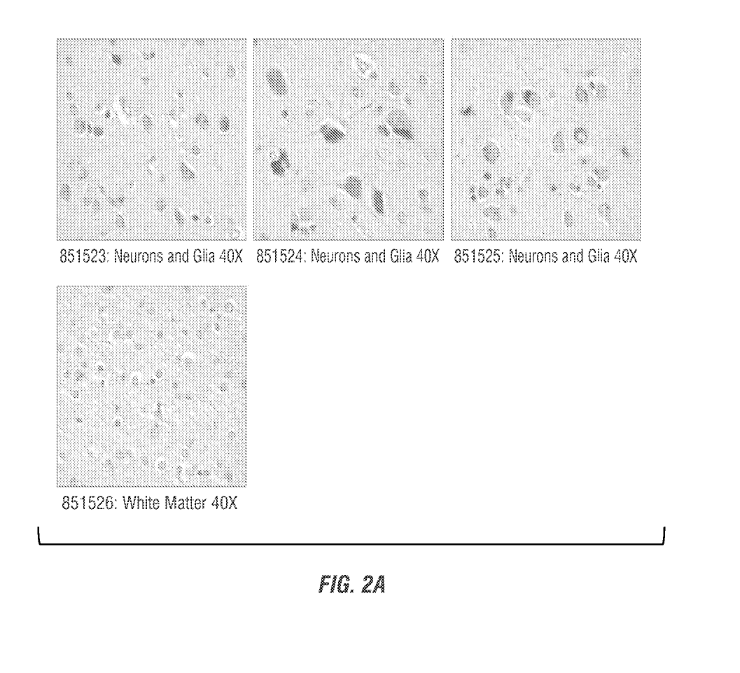

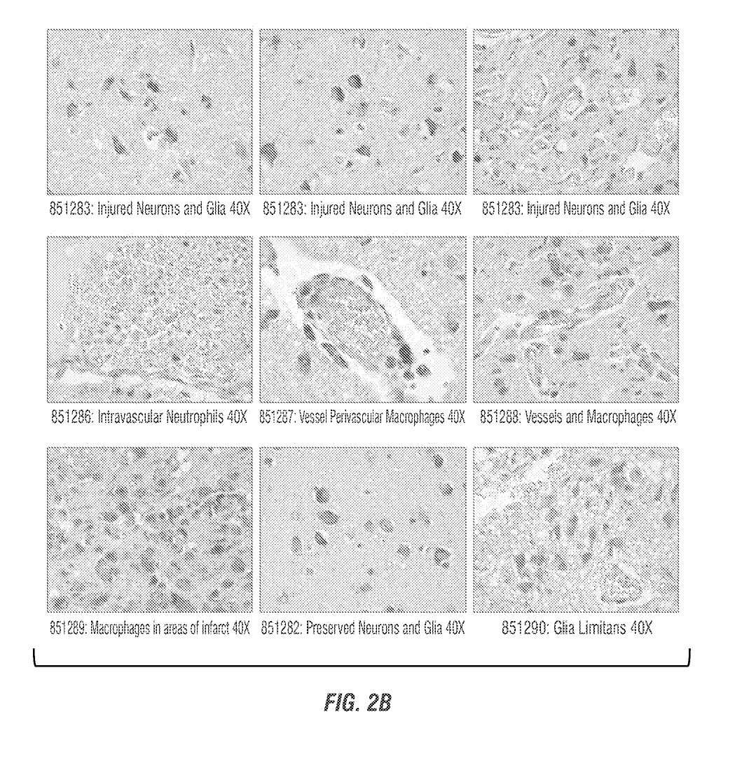

FIG. 2 (A-B) shows samples of cerebral cortex stained for expression of ETT. (A) Cerebral cortex sample obtained from an 84-year-old female with hypertension who died of chronic obstructive pulmonary disease. Sections showed normal cortex. Neurons within all layers of the cortex showed faint to moderate granular cytoplasmic staining, with slightly greater staining of pyramidal and larger neurons. Faint staining was also observed in capillaries, with faint staining of occasional endothelial cells and pericytes. Faint to moderate staining was observed in perivascular macrophages. Within glia, the majority of astrocytes and oligodendroglia within the white matter were negative. Occasional astrocytes or microglia showed rare faint granular staining. (B) Brain tissue sample obtained at autopsy from a 67-year-old male. The sample shows a section of cortex with an infarct encompassing most of the section. In areas away from the infarct, staining of neurons ranged from faint to occasionally moderate. In areas adjacent to the infarct, residual surviving neurons showed moderate to strong staining. Areas of infarction showed moderate staining of macrophages and moderately positive reactive astrocytes. Occasional oligodendroglia also showed faint staining. Microgila were also moderate. Vessels showed faint to moderate staining of endothelium and vascular smooth muscle. Compared to normal brain, this section showed increased staining with neurons adjacent to areas of infarction, and increased staining within macrophages and reactive astrocytes.

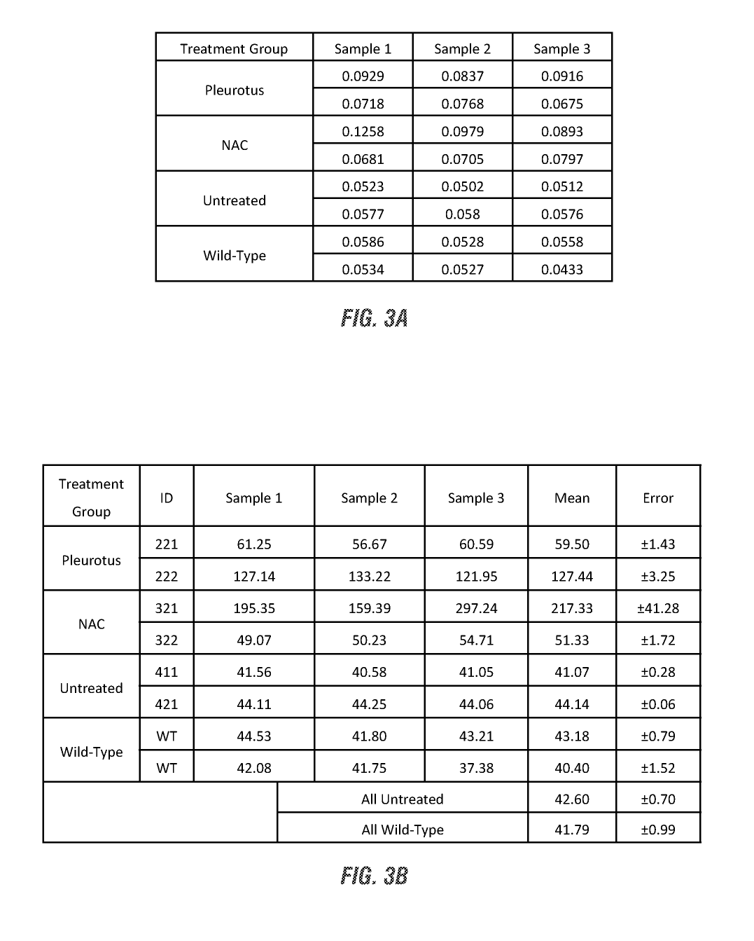

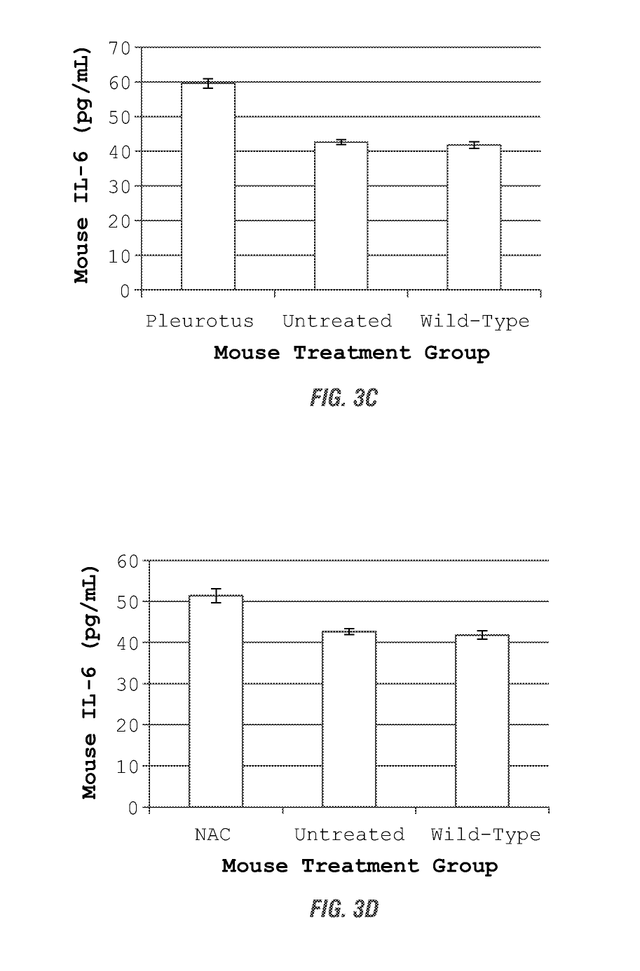

FIG. 3 (A-D) shows the results of IL-6 assays performed on Alpha synuclein transgenic mice treated with ET or NAC. (A) Sample absorbency values (A450-A550), in blood samples from wild-type and Alpha synuclein transgenic mice unadjusted for dilution. (B) Sample IL-6 concentrations (pg/mL) in blood samples from wild-type and Alpha synuclein transgenic mice, adjusted for dilution. (C) IL6 (pg/mL) in blood samples from wild type and Alpha synuclein transgenic mice. Transgenic mice were untreated of dosed with ET (Pleurotus). (D) IL6 (pg/mL) in blood samples from wild type and Alpha synuclein transgenic mice. Transgenic mice were untreated of dosed with NAC.

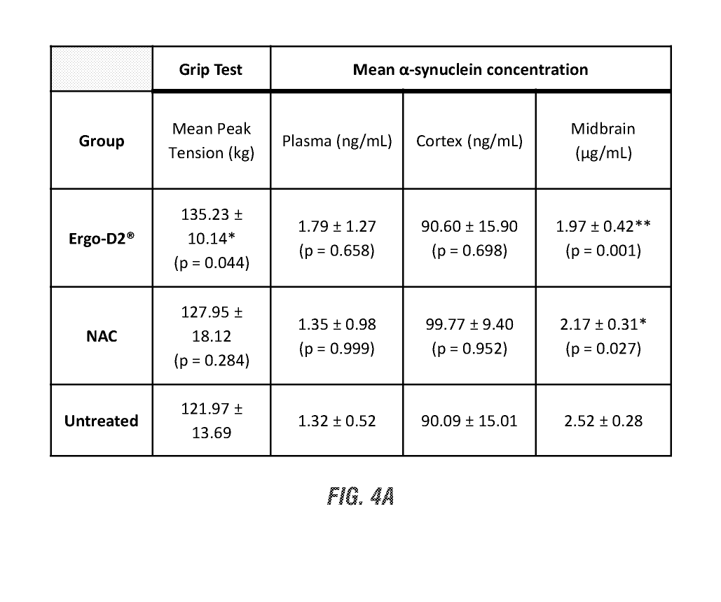

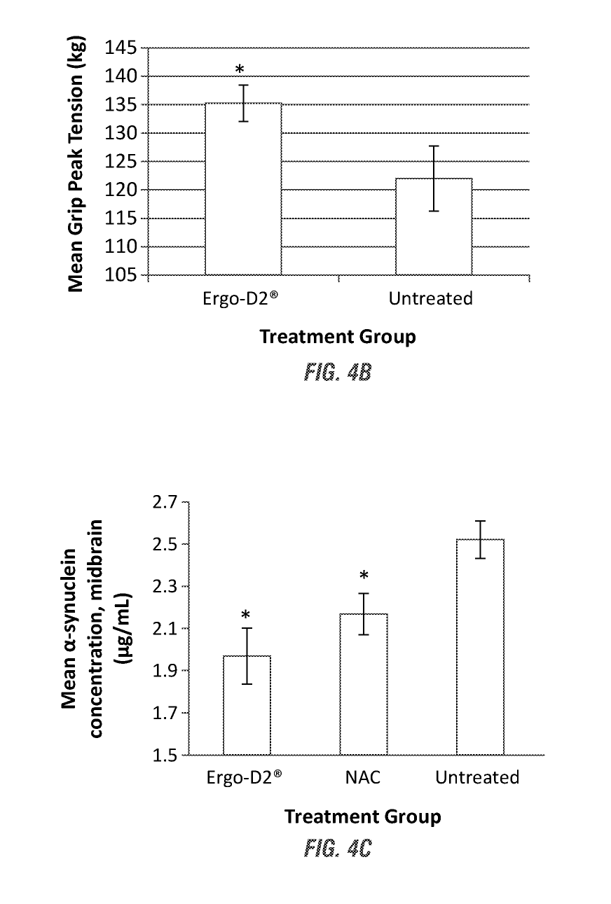

FIG. 4 (A-C) shows grip strength and alpha-synuclein concentrations in a SNCA transgenic mouse model of PD after three months of formulation treatment. (A) Treatment group differences in grip test mean peak tension and plasma, cortex and midbrain α-synuclein concentration. Significance indicated for each treatment group compared to untreated controls. * is significant at the p<0.05 level. ** is significant at the p<0.005 level. Three consecutive trials were performed for each mouse and the average used for statistical analysis. (B) Mean grip peak tension for subjects treated with Ergo-D2® vs. untreated controls. * is significant compared to control group at the p<0.05 level. Error depicted as ±SEM. (C) Mean α-synuclein concentration in midbrain tissue from each treatment group. * is significant compared to untreated controls at the p<0.05 level. ** is significant compared to untreated controls at the p<0.005 level. Error depicted as ±SEM.

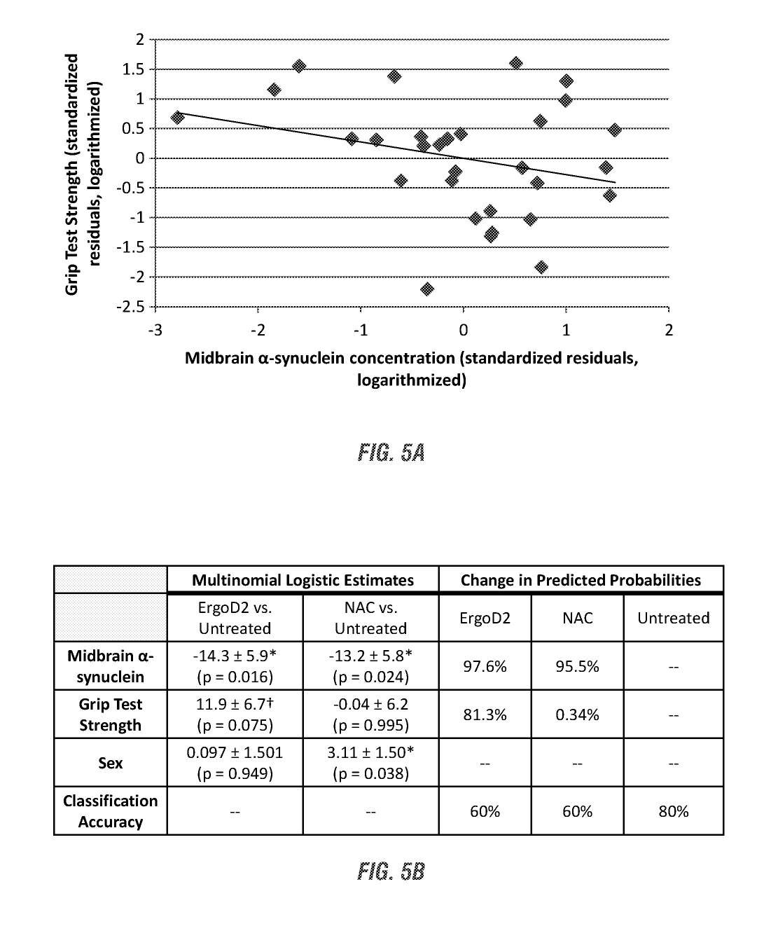

FIG. 5 (A-B) shows the statistical relationship between grip test strength and midbrain α-synuclein concentration in a SNCA transgenic mouse model of PD after three months of formulation treatment. (A) Partial correlation between logarithmized midbrain a-synuclein concentration and grip test strength, controlling for sex. Figure depicts correlation between standardized residuals of each measurement regressed on the sex of the subject mice. r=−0.275, p=0.075. (B) Multinomial logistic regression coefficients, classification probability ranges, and overall classification accuracies between each treatment group and untreated controls, using midbrain α-synuclein concentration and grip test strength (both logarithmized) as predictors while controlling for sex of the mice. Changes in predicted probabilities were calculated between the extreme values of each predictor while holding other predictors and controls constant at their means. † is significant at the p<0.10 level, * is significant at the p<0.05 level (both two-tailed).

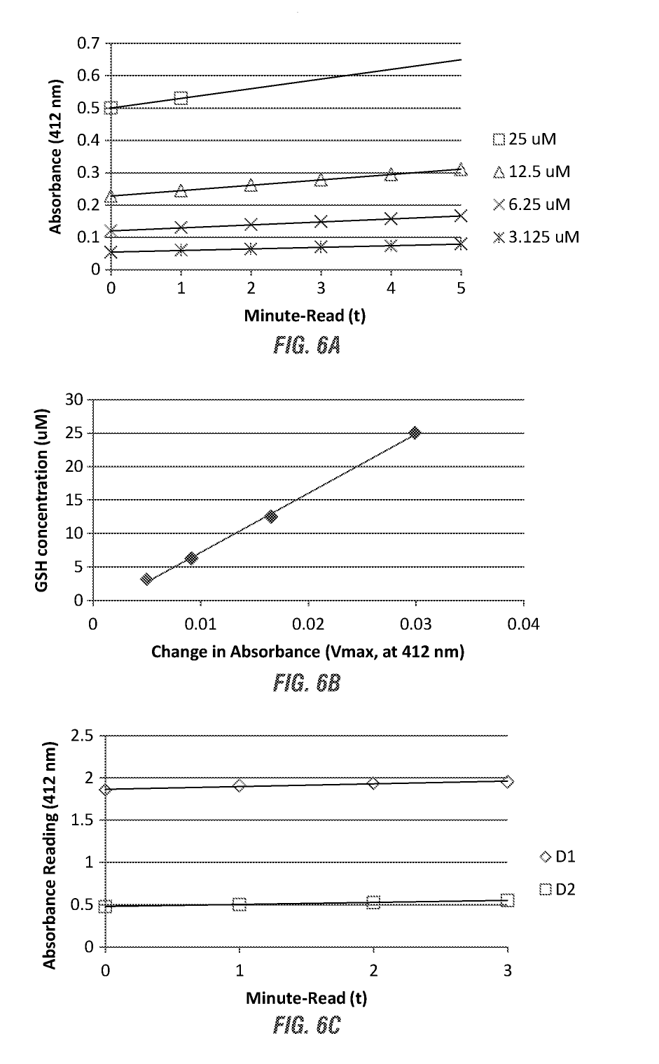

FIG. 6 (A-C) shows kinetic assay protocol for the presence of glutathione (GSH). (A) The slope of each linearly regressed line indicates the change in absorbance, or Vmax, for each sample. Using only the first two minute-reads of the 25 uM standard yields the expected upward-sloping, linear change in absorbance and ultimately allows for the creation of a similarly upward-sloping, linear standard curve. (B) Standard curve formed by plotting change in absorbance (Vmax) of standard solutions against known GSH concentrations. A linear regression gave the following equation: y=884.15x−1.6738 (R2=0.9984). This equation was used to determine the GSH concentrations in the subject plasma samples based on their kinetic absorbance readings (Vmax). (C) Kinetic absorbance readings for the second wild-type subject, in duplicate and omitting the final two reads (t=4 and t=5). Though the actual absorbance values for each duplicate sample (D1 and D2) vary significantly, linear regression of either series of readings yields Vmax values that vary by less than 15% (0.0101 and 0.0123, respectively). This minimal variance and a clear upward-sloping, linear trend for the change in absorbance of each duplicate provide justification for the use of this sample data, even if incomplete.

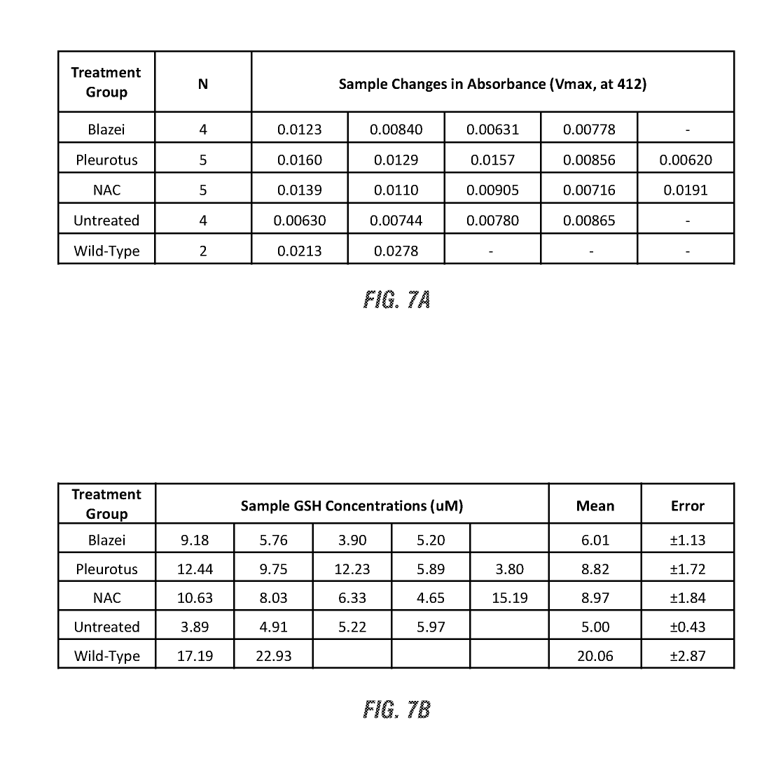

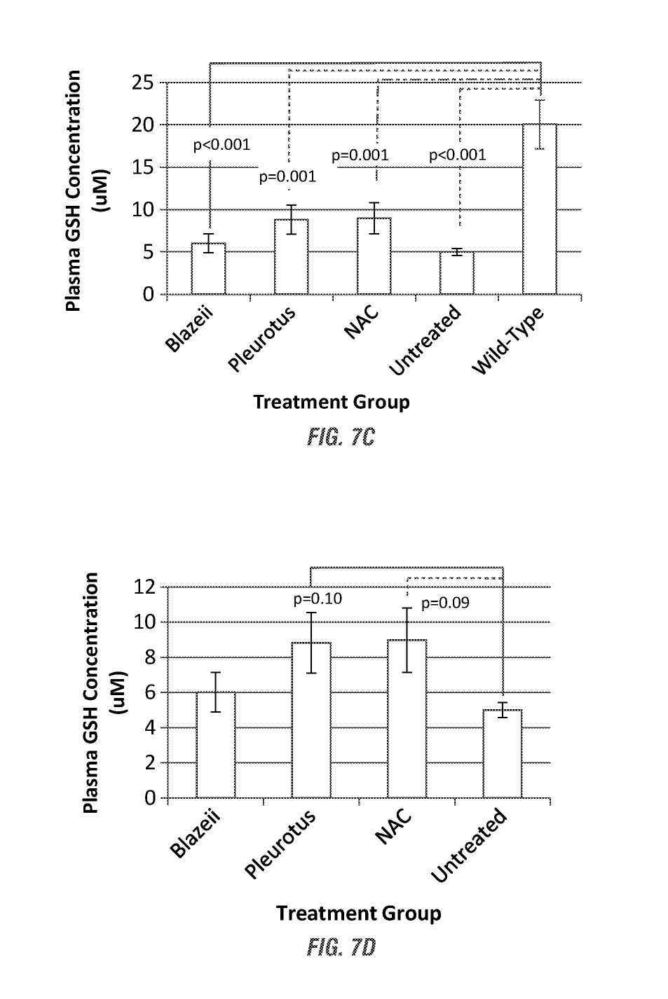

FIG. 7 (A-D) shows detection of glutathione in plasma of Pac-Tg (SNCA) mice transgenic mice. The tested subject groups were treated with one of the following: Blazei Mushroom, Pleurotus mushroom, or n-acetyl cysteine (NAC). (A) Change in absorbance (412 nm) taken during kinetic assay for each plasma sample (Vmax) (B) Sample glutathione (GSH) concentrations (uM), calculated using each Vmax reading and a linear regression of the standard curve. Each sample was prepared in a 1:2 dilution with provided 5% SSA solution, though the assay instructions indicated that this preparation should be treated as original sample (i.e. not accounting for a dilution factor). (C) Calculated plasma glutathione concentrations for each treatment group, including untreated controls and wild-types. Statistically-significant differences between the groups are indicated by lines connecting the bars representing each group, with p-values for each difference listed on the graph. (D) Calculated plasma glutathione concentrations for each treatment group, compared to the untreated control group. Trends toward statistical significance are indicated by lines connecting the bars representing each group, with associated p-values listed on the graph.

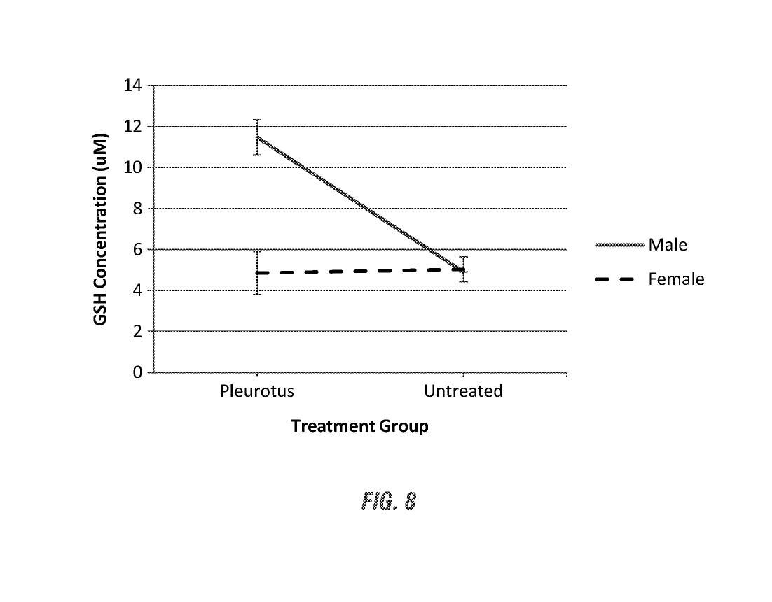

FIG. 8 shows possible interaction between sex and treatment group for Pleurotus-treated subjects compared to untreated controls. An unprotected Fisher post-hoc demonstrates a statistically significant interaction (between p=0.02 and p=0.03), though the low sample sizes (i.e. only one male untreated subject) and lack of statistically significant interaction elsewhere renders this finding highly suspect.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE INVENTION

The embodiments of this invention are not limited to particular embodiments for compositions and uses of Ergothioneine for tissue and organ dysfunction and associated mitochondrial dysfunction and related comorbidities, which can vary and are understood by skilled artisans. It is further understood that the terms cells and tissues include the encompassing organ, and muscles or muscular tissues include somatic, smooth and cardiac muscles. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers defining the range and include each integer within the defined range.

Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities refers to variation in the numerical quantity that can occur.

As used herein the term “anemia” refers to a decrease in number of red blood cells (erythrocytes) or less than the normal quantity of hemoglobin in the blood. Any abnormality in hemoglobin or erythrocytes results in reduced oxygen carrying capacity and reduced oxygen levels in the blood, as well as tissues and organs. Anemia with associated decreased oxygen tension within tissues can also include decreased oxygen-binding capacity of hemoglobin molecules due to deformity or abnormalities of hemoglobin binding of oxygen due to hemoglobin iron in the plus 3 status. The iron atom in the heme group must initially be in the ferrous (Fe2+) oxidation state to support oxygen and other gases' binding and transport. Initial oxidation to the ferric (Fe3+) state without oxygen converts hemoglobin into “hemiglobin” or methemoglobin, which cannot bind oxygen. Hemoglobin in normal red blood cells is protected by a reduction system to keep this from happening. Anemia can also be associated with abnormal production, processing, or performance of erythrocytes and/or hemoglobin. The term anemia refers to any reduction in the number of red blood cells and/or level of hemoglobin in blood relative to normal blood levels. The term anemia as used also refers to the size of red blood cells and size is reflected in the term mean corpuscular volume (MCV). The classifications of anemia using MCV include macrocytic, normocytic and microcytic anemia. Kinetic approaches to defining anemia include analysis of the reticulocyte count which is a quantitative measure of the bone marrow's production of new red blood cells. The degree of anemia is assessed by measuring the reticulocyte production index which is a calculation of the ratio between the level of anemia and the extent to which the reticulocyte count has risen in response. The term transferrin refers to an iron-binding blood plasma glycoprotein that controls the level of free iron in biological fluids. When not bound to iron, it is called apotransferrin. The term ferritin refers to an intracellular protein that stores iron and releases it in a controlled fashion. The amount of ferritin reflects the amount of iron stored and also acts as a buffer against iron deficiency and iron overload.

As one skilled in the art will appreciate, anemia can arise due to a variety of conditions such as acute or chronic kidney disease, infections, inflammation, cancer, irradiation, toxins, diabetes, and surgery. For example, infections may be due to, e.g. virus, bacteria, and/or parasites, etc. Inflammation may be due to acute or chronic trauma, infection, autoimmune disorders, such as rheumatoid arthritis, autoimmune hemolytic anemia, transfusion reactions, etc. One skilled in the art shall appreciate the numerous applications for the compositions and the methods of use disclosed according to the present invention.

As used herein the term “mushroom” or “filamentous fungi” shall be interpreted to include all tissues, cells, organs of the same, including but not limited to mycelium, spores, gills, fruiting body, stipe, pileus, lamellae, basidiospores, basidia, and the like.

As used herein the term “naturally-enhanced” with respect to whole foods such as mushrooms, yeast, cyanobacteria, Spirulina and Vitamin D, shall include pulsed UV irradiated mushrooms, yeast, cyanobacteria, Spirulina, etc. produced by the methods disclosed herein. The naturally-enhanced products according to the invention may include the enhanced whole food as well as powders and other forms obtained from the whole food.

The terms “subject” or “patient” are used herein interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

The term “treating” or “treatment” as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” or “treatment” includes the administration of the compounds or agents of the present invention which may be in combination with other compounds.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

Compositions

According to an embodiment of the invention, a nutritional supplement, ingredient, food or beverage composition and/or pharmaceutical composition for treating anemia and preventing the comorbid disease states associated therewith may include Ergothioneine, Vitamin D2 and/or D3, phytonutrients, beta glucans, omega-3 or alternative antioxidants, a pharmaceutically-acceptable carrier and/or combinations of the same.

As used herein the term Ergothioneine shall be interpreted to include variants, homologs, optical isomers and the like which retain the antioxidant activity of Ergothioneine or L-Ergothioneine as demonstrated and described herein. Ergothioneine is a naturally-occurring amino acid. Ergothioneine is a natural antioxidant but is unable to be made in human cells; rather it is absorbed from the diet. Ergothioneine from any suitable source may be used according to the invention. L-Ergothioneine is available commercially from Oxis International, Inc., Sigma Chemical, etc. or from dietary sources such as mushrooms and the various sources disclosed herein according to the invention. The compound is also available from Actinobacteria, filamentous fungi, cyanobacteria, Spirulina, oats, barley and other whole food sources. Ergothioneine for use in compositions according to the invention may be obtained from an independent bionutrient source, such as Vitamin D enriched mushrooms disclosed herein, whole food sources, cyanobacteria and Spirulina as disclosed according to the embodiments of the invention.

According to one embodiment of the invention, Vitamin D2 and/or D3 may be provided from a UV irradiated, Agaricus fungi, tissue, substrate or component thereof with higher levels of Vitamin D2 than a non-irradiated product. According to an embodiment of the invention, the novel mushroom whole food (Ergo-D2), obtained from Agaricus fungi but also other fungi, such as Pleurotus eryngii, may be used. Ergo-D2 contains high levels of three bioactive components previously shown to have health promoting properties-Vitamin D2, L-Ergothioneine (ET) and beta-glucans.

Vitamin D and Ergothioneine enriched mushrooms according to the invention are pulsed with UV light at lower ranges and for very brief periods have increases by as much as 800 times the % DV (percent daily value) of Vitamin D content, per serving with no deleterious effects on the morphology or appearance of the mushroom. Pulsed UV-light treatments to increase Vitamin D₂ content in mushrooms were conducted with a laboratory scale, pulsed light sterilization system (SteriPulse®-XL 3000, Xenon Corporation, Woburn, Mass.) that is present in the Department of Agricultural Biological Engineering at Pennsylvania State University, State College, Pa. While applicants postulate that it is the UVB component of the Xenon pulsed light system that is responsible for the effects of the invention, it should be noted that the system uses pulsed light which includes the entire spectrum of light and may also include other components that contribute to the effects demonstrated herein and which are intended to be within the scope of the invention.

Any type of mushroom, mushroom part, component, fungi or even used substrate for cultivating mushrooms, with ergosterol present may be used. This includes all filamentous fungi where ergosterol has been shown to be present and includes the use of tissues such as the mycelia, spores or vegetative cells. This includes, but is not limited to, for example, Coprinus, Agrocybe, Hypholoma, Hypsizygus, Pholiota, Pleurotus, Stropharia, Ganoderma, Grifola, Trametes, Hericium, Tramella, Psilocybe, Agaricus, including for example Agaricus bisporus (e.g. white button mushrooms), Phytophthora achlya, Flammulina, Melanoleuca, Agrocybe, Morchella, Mastigomycotina, Auricularia, Gymnopilus, Mycena, Boletus, Gyromitra, Pholiota, Calvatia, Kuegneromyces, Phylacteria, Cantharellus, Lactarius, Pleurotus, Clitocybe, Lentinula (Lentinus), Stropharia, Coprinus, Lepiota, Tuber, Tremella, Drosophia, Leucocoprinus, Tricholoma, Dryphila, Marasmius, and Volvariella.

In addition, the solid substrate can be any part of the mushroom or mold, including the mycelia, spores etc., so long as ergosterol is present in at least part of the tissue or cells. In yet another embodiment, the spent mushroom substrate upon which mushrooms are cultivated, was enriched in Vitamin D using pulsed UV light according to the invention. As one skilled in the art shall ascertain, mushrooms are usually produced by first preparing a substrate, such as corn, oats, rice, millet or rye or various combinations, prepared by soaking the grain in water and sterilizing the substrate before inoculation with mushroom spores or mushroom mycelia. Mycelia are the filamentous hyphae of a mushroom that collect water and nutrients to enable mushrooms to grow. The inoculated substrate is then held to promote colonization of the mycelia, at which point the mycelia-laced grains become “spawn”. This is usually done in individual spawn bags. The substrate provides the nutrients necessary for mycelium growth. The mycelium-impregnated substrate then develops under controlled temperature and moisture conditions, until the hyphae of the mycelium have colonized the substrate. The mycelium enriched product usually is harvested after about four to eight weeks from the beginning of the process, with the contents of the spawn bag possibly processed into dry powdered product. According to the invention, this spent substrate may also be enriched in Vitamin D upon application of pulsed UV irradiation.

Non-limiting examples of other fungal genera, including fermentable fungi, include: Alternaria, Endothia, Neurospora, Aspergillus, Fusarium, Penicillium, Blakeslea, Monascus, Rhizopus, Cephalosporium, Mucor, and Trichoderma.

In addition to the irradiated mushrooms according to an embodiment of the invention for providing a composition with enhanced Ergothioneine and Vitamin D, additional substrates for Ergothioneine may be irradiated to enhance the Ergothioneine content, including for example cyanobacteria and Spirulina. According to a further embodiment of the invention, cyanobacteria and/or Spirulina may be added as an additive ingredient to the irradiated mushrooms. According to a further embodiment of the invention, cyanobacteria and/or Spirulina may be irradiated and added to irradiated mushrooms.

Additional antioxidants may be beneficial in the compositions according to the invention. For example, turmeric and its active component curcumin, are phytonutrients that act as antioxidants. According to an embodiment, the compositions of the invention comprise a phytonutrient antioxidant in addition to the fungi component to provide a combined synergistic response.

Phytonutrients according to the invention include natural n-acetyl cysteine (NAC), turmeric, monophenols, flavonoids, polyphenols, phenolic acids, hydroxycinnamic acids, lignans, phytoestrogens, tyrosol esters, stilbenoids, punicalagins, alkylresorcinols, carotenoids, tetraterpenoids, monoterpenes, saponins, phytosterols, tocopherols, omega-3, 6,9 fatty acids, oleanolic acid, ursolic acid, betulinic acid, moronic acid, betalains, dithiolthiones, isothiocyanates, polysulfides, sulfides, indole-3-carbinol, sulforaphane, 3,3′-Diindolylmethane, sinigrin, allicin, alliin, allyl isothiocyanate, piperine, syn-propanethial-S-oxide, oxalic acid, phytic acid, tartaric acid, and anacardic acid.

An example of a suitable phytonutrient according to the invention is turmeric. Turmeric is available in various forms contains up to 5% essential oils and up to 5% curcumin, a polyphenol. Curcumin is the active substance of turmeric and curcumin is known as C.I. 75300, or Natural Yellow 3. The systematic chemical name is (1E,6E)-1,7-bis(4-hydroxy-3-methoxyphenyl)-1,6-heptadiene-3,5-dione and exists in tautomeric forms—keto and enol.

Food or Beverage Compositions

An embodiment of the present invention also provides natural bionutrients, medical foods and/or beverages comprising combinations of Ergothioneine, enriched mushrooms of the invention including extracts, fractions thereof or compounds thereof or any combination thereof, phytonutrients and/or antioxidants. The food compositions according to the invention may comprise enriched mushrooms from a variety of fungi sources as disclosed according to embodiments herein this description. Alternatively, the food compositions according to the invention may comprise Ergothioneine obtained directly from whole food sources, Spirulina or cyanobacteria.

The medical food is compounded for the amelioration of a disease, disorder or condition associated with or caused by inflammation, oxidative stress and/or decreased levels of Ergothioneine. According to a preferred embodiment of the invention, food compositions are intended for human consumption for daily supplementation. Ranges of the amounts of each component of the food compositions can be adjusted as necessary for the supplementation of individual patients and according to the specific condition treated. Any variations in the amount of the ingredients may be utilized according to the desired composition formulation.

The medical foods according to the invention are formulated to manage a specific disease or condition for which medical evaluation, based on recognized scientific principles, has established distinct nutritional requirements. All components of the medical foods have GRAS status (Generally Recognized as Safe) as designated by the FDA or independent review. In a preferred embodiment, a medical food according to the invention, ErgoD2™ Hemo, is an encapsulated medical food that is certified organic and may be dispensed by a medical practitioner as indicated for the distinct nutritional requirements of patients being treated for diabetes and/or anemia, as disclosed herein according to the methods of the invention.

The food composition according to the invention may be prepared by any of the well-known techniques known by those skilled in the art, consisting essentially of admixing the components, optionally including one or more accessory ingredients. In one embodiment, the extracts, fractions, and compounds of this invention may be administered in conjunction with other additives and fillers known to those of skill in the art. Other compatible actives may be included in the food compositions of the present invention.

According to one embodiment of the invention, a beverage composition is provided. For particularly suitable applications for patients suffering from anemia, such as dialysis patients, a beverage composition is provided on a daily basis. According to a further embodiment, a food supplement is provided on a daily basis, to ensure that the supplementation provides a whole food source of the Ergothioneine and Vitamin D. Although not intended to be limited according to a particular theory of the present invention, providing a whole food source administers various co-enzyme factors from the whole food providing additional supplementation and treatment benefits. According to an alternative embodiment, an extracted source of the Ergothioneine and Vitamin D (e.g. dried mushroom powder or Spirulina) can be added to the food or beverage composition.

Pharmaceutical Compositions

In an embodiment of the invention, a pharmaceutical composition for treating a disease state including anemia or diabetes. In an additional embodiment, a pharmaceutical composition for treating a disease state associated with inflammation, oxidative stress and/or decreased levels of Ergothioneine comprises a combination of the following ingredients (in a variety of combinations, such that not every component is required according to various embodiments of the invention), a source of Ergothioneine, a UV irradiated, enriched mushroom, tissue, substrate or component thereof with higher levels of Vitamin D2 than a non-irradiated product, and a pharmaceutically-acceptable carrier. The pharmaceutical compositions according to the invention may further comprise antioxidants, phytonutrients and other beneficial components for treatment of the conditions disclosed herein.

According to a further embodiment of the invention, the pharmaceutical composition may further comprise another bioactive nutrient attached to Ergothioneine. Although not intended to be limited to a particular theory of the invention, the attachment of a bionutrient to Ergothioneine delivers the bionutrient along with the Ergothioneine, wherein the Ergothioneine acts as an active carrier to deliver the bionutrient to a cell. According to an additional non-limiting theory of the invention, the ETT permits the bionutrient to enter the cell. For example, selenium and/or extracted products from beer hops, oats, barley, etc. can be added to the Ergothioneine and the pharmaceutical compositions of the invention.

The pharmaceutically-acceptable carrier according to the invention facilitates administration of the composition to a patient in need thereof. The turmeric, Ergothioneine and the compound, extracts, fractions and/or compounds derived from the enriched mushrooms of the invention may be mixed with any of a variety of pharmaceutically-acceptable carriers for administration. “Pharmaceutically acceptable” as used herein means that the extract, fraction thereof, or compound thereof or composition is suitable for administration to a subject to achieve the treatments described herein, without unduly deleterious side effects in light of the severity of the disease and necessity of the treatment. According to the invention, the carrier may be a solid or a liquid, or both, and is preferably formulated with the compound as a unit-dose formulation, for example, a tablet, which may contain from 0.5% to 95% by weight of the active compound.

The pharmaceutical composition according to the invention may be prepared by any of the well-known techniques of pharmacy consisting essentially of admixing the components, optionally including one or more accessory ingredients. In one embodiment, the extracts, fractions, and compounds of this invention may be administered in conjunction with other medicaments known to those of skill in the art. Other compatible pharmaceutical additives and actives may be included in the pharmaceutically acceptable carrier for use in the compositions of the present invention.

Dose ranges of the pharmaceutical compositions can be adjusted as necessary for the treatment of individual patients and according to the specific condition treated. Any of a number of suitable pharmaceutical formulations may be utilized as a vehicle for the administration of the compositions of the present invention and maybe a variety of administration routes are available. The particular mode selected will depend of course, upon the particular formulation selected, the severity of the disease, disorder, or condition being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, transdermal or parenteral routes and the like. Accordingly, the formulations of the invention include those suitable for oral, rectal, topical, buccal, sublingual, parenteral (e.g., subcutaneous, intramuscular, intradermal, inhalational or intravenous) and transdermal administration, although the most suitable route in any given case will depend on the nature and severity of the condition being treated and on the nature of the particular active product used.

Formulations suitable for oral administration may be presented in discrete units, such as capsules, cachets, lozenges, drops, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, inhalational or intradermal injection. Such preparations may conveniently be prepared by admixing the compound with water or a glycine buffer and rendering the resulting solution sterile and isotonic with the blood. Alternately, the extracts, fractions thereof or compounds thereof can be added to a parenteral lipid solution.

Formulations of the inventive mixtures are particularly suitable for topical application to the skin and preferably take the form of an ointment, cream, lotion, paste, gel, spray, aerosol, or oil. Carriers which may be used include Vaseline, lanoline, polyethylene glycols, alcohols, transdermal enhancers, and combinations of two or more thereof.

Formulations suitable for transdermal administration may also be presented as medicated bandages or discrete patches adapted to remain in intimate contact with the epidermis of the recipient for a prolonged period of time. Formulations suitable for transdermal administration may also be delivered by iontophoresis (passage of a small electric current to “inject” electrically charged ions into the skin; also called electromotive drug administration (EMDA)) through the skin. For this, the dosage form typically takes the form of an optionally buffered aqueous solution of the active compound. Suitable formulations comprise citrate or bis/tris buffer (pH 6) or ethanol/water and contain from 0.01 to 0.2M active ingredient.

The therapeutically effective dosage of any specific compound will vary somewhat from compound to compound, patient to patient, and will depend upon the condition of the patient and the route of delivery. As a general proposition, a dosage from about 0.01 to about 50 mg/kg will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight or volume of the enriched mushrooms, fractions thereof or compounds thereof of the present invention, including the cases where a salt is employed. In an aspect of the invention a pharmaceutical composition provided in 500 mg capsules may be dosed to a patient from 1 to 4 capsules a day, preferably 2 to 4 capsules a day.

In an aspect of the invention, the pharmaceutical composition provides a blend of mushroom antioxidants and optionally phytonutrients. In certain aspects, the pharmaceutical composition may be classified also as a medical food. High concentrations of natural Ergothioneine and Ergocalciferol (vitamin D2) are included in the compositions for administration to a patient in need thereof. In an aspect of the invention, the compositions may be formulated as vegan products. In an additional aspect of the invention, the compositions contain USDA certified organic ingredients and do not include any artificial colors, flavors, or preservatives. In a further aspect of the invention, the compositions provide a natural, non-toxic product.

Extraction of Ergothioneine from Various Sources for Use in Compositions of the Invention

The isolation, extraction and/or sourcing of Ergothioneine from additional sources is disclosed according to the methods of use of the present invention. As a result, various whole sources of food and/or bacteria may be used to provide the Ergothioneine required for the methods of use and/or the compositions according to the invention.

Previously the extraction of Ergothioneine was achieved from the enriched mushroom sources disclosed herein. The mushrooms were further enriched with Vitamin D2 and/or D3 and could be obtained, for example, from a UV irradiated, Agaricus fungi, tissue, substrate or component thereof with higher levels of Vitamin D2 than a non-irradiated product. A preferred source for the enriched mushroom is the whole food (Ergo-D2), containing high levels of three bioactive components: Vitamin D2, L-Ergothioneine (ET) and beta-glucans.

According to a further embodiment of the invention, Ergothioneine can further be obtained from cyanobacteria. Cyanobacteria can be used for extraction of Ergothioneine and/or a source for Ergothioneine. Spirulina is blue-green algae that have been identified to be a source of Ergothioneine. Spirulina is a microscopic blue-green alga in the shape of a spiral coil, living both in sea and fresh water. It is the most common name for human and animal food or nutritional supplement made primarily from two species of cyanobacteria: Arthrospira platensis and Arthrospira maxima.

According to a further embodiment, various plant materials are used to source Ergothioneine for the methods of use and/or the compositions according to the invention.

Plant material sources for Ergothioneine may include cereal grains, including oats, wheat and barley. Ergothioneine may be further extracted from beer hops, and cereal grains, including oats, barley, etc.

Upon extraction or isolation of Ergothioneine from a source additional molecules and entities can be attached to permit delivery into the cell along with the Ergothioneine. As is recognized in the art relating to Ergothioneine, the Ergothioneine transporter (ETT) provides a mechanism of delivery of Ergothioneine within cells. As a result, it is desirable to attach additional molecules to Ergothioneine, upon isolation from at least the sources disclosed herein (e.g. whole foods and cyanobacteria), including for example, beta-glucans, antioxidants, selenium, phytonutrients, and/or vitamins, such as Vitamin C and Vitamin D2. The attachment of additional molecules to an extracted source of Ergothioneine permits the effective delivery into the mitochondria of the cells of a patient in need of treatment according to the embodiments of the invention.

Enhancement and Extraction of Vitamin D2 and Other Bioactive Ergosterol-Derived Products Following Pulsed UV Light Exposure of Mushrooms from Various Sources for Use in Compositions of the Invention.

Exposure of mushrooms to UV light irradiation generates, in addition to Vitamin D2, additional ergosterol derived products, such as pre-vitamin D2, lumisterol2 and tachysterol. Vitamin D2 is the most abundant product, followed by pre-vitamin D2, lumisterol2 and tachysterol2 (order of decreasing abundance). In addition, untreated mushroom samples did not contain detectable levels of any photoproduct. (Kalaras, M. D., Beelman, R. B., Elias, R. J., Effects of postharvest pulsed UV light treatment of white button mushrooms (Agaricus bisporus) on vitamin D2 content and quality attributes. Journal of Agricultural and Food Chemistry (2012), 60 (1): 220-225). This reference is herein incorporated by reference in its entirety.

As an embodiment of this invention, the use of UV enhanced mushrooms, including ErgoD2™ medical food, and/or extracts and the resultant physiologic effects may be associated not only with Vitamin D2 but also with ergosterol derived photoproducts.

Methods of Use—Muscle Cell and Tissue Function, Strength, Flexibility Endurance, Repair and Protection

Embodiments of the invention include methods of improving muscle, cell and tissue function, strength, endurance, repair, protection and associated mitochondrial function within muscles. The methods of use disclosed herein may be used to decrease inflammation within muscles and increase the resistance of muscle tissue and cells to oxidative stress and release of toxic free radicals within muscles as part of their normal metabolic process and physiologic function.

Without being limited to a particular theory of the invention, the use of the compositions for treatment and/or improvement of cellular, tissue and organ function, including muscular function supplies electrons to stabilize and reactivate hemoglobin, in addition to maintaining iron in the +2 oxidation state required for oxygen binding. Furthermore, the use of these compositions for treatment and/or improvement of cellular, tissue and organ function, including muscular function involves the mobilization of iron from sequestered functional iron pools with associated increases in blood iron and total percentage iron saturation levels. The treatments according to the invention may also stimulate progenitor bone marrow stem cells to increase production of red cells. These and other benefits of using Ergothioneine and/or the various compositions according to the invention indicate the usefulness in improving muscle function, strength and endurance. Moreover, the ability of the antioxidant ergothioneine to neutralize free radicals allows its use in autoimmune neuromuscular disorders such as multiple sclerosis and Guillan-Barre syndrome. MS is associated with iron deficiency and the ability of ErgoD2™ to increase blood levels of iron suggests a potential application for ErgoD2™ in this condition.

In a further aspect of the invention, the methods revealed demonstrate improved iron levels, and increases in percentage iron saturation, blood cell counts and hemoglobin levels.

The improved method of improving muscle metabolism and function using Ergothioneine according to the invention may also be administered in combination with use of additional supportive antioxidants such as Vitamin D2, n-acetyl cysteine (NAC), turmeric, Vitamin C, glutathione, etc. Enhanced supplements, compositions and/or pharmaceutical preparations for treating conditions associated with oxidative stress and inflammation, such as autoimmune diseases and anemia, are also disclosed.

According to an embodiment of the invention, a method of treating anemia, including iron deficiency, comprises administering to an animal or patient in need thereof a source of Ergothioneine and a naturally extracted and/or enhanced source of Vitamin D, wherein upon administration of the same improves the treatment of anemia and/or deficient iron levels. According to an embodiment of the invention, the enhanced source of Vitamin D may be obtained from a filamentous fungi, tissue, substrate, spent substrate or component thereof, with increased levels of Vitamin D. A suitable example is the novel mushroom whole food ErgoD2™ (see Table 3).

Beneficial effects of treatment according to the invention were obtained using dosage as set out in Table 4. Thus, in an embodiment of the invention, Ergothioneine is provided at least at 0.5 μg/35 g body weight. In a more preferred embodiment, Ergothioneine is provided from about 0.5 μg/35 g body weight to about 5 g/35 g body weight. In a more preferred embodiment, Ergothioneine is provided from at about 1.5 μg/35 g body weight.

In another embodiment of the invention, Vitamin D2 is provided at least at 1 IU/35 g of body weight. In a preferred embodiment of the invention, Vitamin D2 is provided at between 1 IU/35 g body weight to 10 IU/35 g body weight. In a more preferred embodiment, Vitamin D2 is provided at about 2.3 IU/35 g body weight.

In a preferred embodiment of the invention, a product such as ErgoD2™ (see Table 3) is provided at least at 0.5 g/35 g body weight. In a preferred embodiment, a product such as ErgoD2™ is provided at about 1 mg/35 g of body weight.

Experimental evidence points to iron serving as a cofactor for muscle function (Carson, J. L. and Adamson, J. W., Iron Deficiency and Heart Disease: Ironclad Evidence?, Abstract, Hematology Am Soc Hematol Educ Program (2010); 2010:348-50). Myoglobin is an iron- and oxygen-binding protein found in the muscle tissue of vertebrates in general and in almost all mammals. According to an embodiment of the invention, a method of treating anemia, including iron deficiency, and also associated muscle dysfunction, poor physiologic function and comorbid muscle diseases involves the administration of the medical food ErgoD2™.

Inflammation and release of toxic free radicals, such as cytokines, have a negative effect on muscular function. As a result, an embodiment of the invention includes supplementation and restoration of Vitamin D levels, in association with the presence of Ergothioneine in order to stimulate production of neutralizing protective antioxidants. These activities associated with the increased presence of ETT in red and white blood cell membranes, including mitochondria, enable L-Ergothioneine (ET) to protect erythrocytes against damage related to HbFeIV O (Grundemann, D. H. et al, Discovery of the Ergothioneine Transporter. Proc. Natl. Acad. Science (PNAS, 2005), 102(14): 5256-5261; Grigat, S. et al, (2007). Probing the Substrate Specificity of the Ergothioneine Transporter with Methimazole, Hercynine, and Organic Cations. Biochem. Pharmacol. (2007), 74: 309-316).

Cells that are dividing rapidly are said to be proliferating. Differentiation results in the specialization of cells for specific functions, such as the production of red blood cells by reticulocytes. In general, differentiation of cells leads to a decrease in proliferation. While cellular proliferation is essential for growth and wound healing, uncontrolled proliferation of cells with certain mutations may lead to diseases like cancer. The active form of vitamin D, 1,25-dihydroxyvitamin D, inhibits proliferation and stimulates the differentiation of cells. (Holick M. F., Vitamin D: importance in the prevention of cancers, type 1 diabetes, heart disease, and osteoporosis, Am J Clin Nutr. (2004) 79(3):362-71).

Methods of use according to the invention may include administration of the compositions, food products, supplements and/or pharmaceutical compositions on a daily basis, weekly basis, or other frequency for the particular purpose. Although not intending to be limited to a particular theory of the invention, it is believed that daily administration of the Ergothioneine and Vitamin D sources benefit more normal muscular function with resultant improvement in muscle strength. Daily supplementation is preferred for those performing strenuous physical exercise and other activities associated with sudden increase stress to muscular tissues. so that they are preloaded with the bionutrients and have elevated serum levels in order to protect against acute and chronic effects of the conditions. Supplementation on a regular and/or daily basis can also build up storage levels of the key bionutrients which can be mobilized at a time of sudden and/or increased physiologic need.

The methods of use disclosed herein may also be used for treating muscle dysfunction, autoimmune disorders associated with muscle dysfunction, and insulin resistance that leads to muscular cell damage and decreased function. Scientists have hypothesized that a person's innate immune response creates an internal inflammatory response in fat tissue, liver and muscle which leads to insulin resistance and muscle dysfunction. In addition, insulin resistance is linked closely to inflammation within tissues and organs. (Shoelson S. E., Lee J., et al., Inflammatory cytokines and the risk to develop type 2 diabetes, J Clin Invest. (2006); 116(1):115-24). In addition, there may be additional or alternative macrophages in people who develop insulin resistance. Macrophages in the adipose tissue, liver, and muscle, as part of innate immunity, secrete pro-inflammatory mediators, creating an inflammation/insulin resistance axis.

The methods discussed herein according to the invention beneficially demonstrate modification of the immune macrophage inflammatory response in the liver through the treatment with compositions of the invention, such as a medical food. In an aspect, a medical food composition, such as ErgoD2™ (see Table 3), according to the invention preserves essential innate immune functions while at the same time decreasing insulin resistance. Treatment according to the invention provides natural bionutrients, including ergothioneine and Vitamin D2, have the ability to inactivate inflammatory signaling molecules (i.e. cytokines or free radicals) which are a major contributing factor in insulin resistance. In an aspect of the invention, the methods discussed result in improved insulin sensitivity and associated improvement in muscle function, strength, and increased athletic performance. In addition, there is an increase in adiponectin levels which are responsible for regulating glucose metabolism and fatty acid catabolism.

Methods of use according to the invention may include administration of the compositions, food products, supplements and/or pharmaceutical compositions on a daily basis, weekly basis, or other frequency for the particular purpose. Although not intending to be limited to a particular theory of the invention, daily supplementation, including multiple doses per day is preferred, so that a patient is preloaded with the bionutrients and maintains elevated serum levels in order to protect against acute and chronic effects of the conditions. Supplementation on a regular and/or daily basis can also build up storage levels of the key bionutrients which can be mobilized at a time of physiologic need.

Methods of Use—Inflammation

The methods of use disclosed herein may be used for various inflammatory diseases and/or conditions associated therewith. According to a further embodiment of the invention, a method of decreasing inflammation within cells, tissues and organs and increasing resistance to oxidative stress and associated disease states comprises administering an effective amount of Ergothioneine and a naturally extracted and/or enhanced source of Vitamin D, such as filamentous fungi that has been naturally enriched in Vitamin D2.

Beneficial effects of treatment according to the invention were obtained using dosage as set out in Table 4. Thus, in an embodiment of the invention, Ergothioneine is provided at least at 0.5 μg/35 g body weight. In a more preferred embodiment, Ergothioneine is provided from about 0.5 μg/35 g body weight to about 5 g/35 g body weight. In a more preferred embodiment, Ergothioneine is provided from at about 1.5 μg/35 g body weight.

In another embodiment of the invention, Vitamin D2 is provided at least at 1 IU/35 g of body weight. In a preferred embodiment of the invention, Vitamin D2 is provided at between 1 IU/35 g body weight to 10 IU/35 g body weight. In a more preferred embodiment, Vitamin D2 is provided at about 2.3 IU/35 g body weight.

In a preferred embodiment of the invention, a product such as ErgoD2™ (see Table 3) is provided at least at 0.5 g/35 g body weight. In a preferred embodiment, a product such as ErgoD2™ is provided at about 1 mg/35 g of body weight.

A still further embodiment of the invention includes a method of treating a physiologic dysfunction and/or a disease state associated with inflammation and/or oxidative stress, including increased production of free radicals comprising administering a composition comprising Ergothioneine and a pulsed UV irradiated, filamentous fungi, tissue, substrate, spent substrate or component thereof, with increased levels of Vitamin D2, wherein upon administration of the same, survivability is increased when compared to an animal with such disease state without such treatment. According to each of the embodiments of the invention the Ergothioneine may be obtained from the whole food sources and/or algae, such as cyanobacteria and Spirulina, as disclosed in this specification.

Demonstrated Efficacy

Applicants demonstrate in animal studies a trend towards an increase in IL-6 concentration between the untreated and treated, especially with NAC. According to an aspect of the invention, IL-6 concentration may increase in response to ErgoD2™ and/or NAC-based therapies to offset pro-oxidant targeting to generate this abnormal protein expression as occurs in neurodegenerative disease conditions and also from acute and chronic stress.

In an aspect of the invention, Ergothioneine-based therapies that increase IL-6 can induce an adaptive ‘acute phase response’ to help the body better cope with stress. Muscle-derived IL-6 is released into the circulation in large amounts and is likely to work in a hormone-like fashion, exerting its effect on the liver and adipose tissue, thereby contributing to the maintenance of glucose homeostasis during exercise and mediating exercise-induced lipolysis. Muscle-derived IL-6 may also work to inhibit the effects of pro-inflammatory cytokines such as TNFα, thereby protecting against insulin resistance and atherogenesis (Pedersen, B. K., et al., Muscle-derived interleukin-6: possible biological effects, J. of Physiol. (2001), 536:329-337).

The test results set forth in Examples 12-17 demonstrate that Ergo-D2 products (from pleurotus mushrooms) and/or additional bioactive products such as NAC increase levels of IL-6 in the whole blood cells of mice. ELISA testing was also performed.

Applicants demonstrated the use of Ergo-D2, a potent anti-oxidant, anti-inflammatory nutritional product, to increase numbers and quality of red blood cells, mean corpuscular hemoglobin concentration (MCHC), and blood iron levels, including total percentage iron saturation.

Applicants demonstrated that the combination of antioxidants, including phytonutrient turmeric and Ergothioneine, along with Vitamin D enriched mushrooms increase longevity in Drosophila kept under nutritionally deficient diet. These results represent a novel use of the compositions of the invention for treating a variety of disease states associated with inflammation and oxidative stress. According to the invention, Applicants have shown that the compositions increase survival and decrease biologic death in conditions associated with oxidative stress, which include disease states such as Alzheimer's disease and other associated diseases including those involving chronic markers of inflammation, such as chronic depression, traumatic brain injury and PTSD. Thus the supplements, food compositions and pharmaceutical compositions according to the invention, employing the Vitamin D enriched mushrooms and Ergothioneine have surprising benefits for treatment of such disease states.

Beneficial effects of treatment according to the invention were obtained using dosage as set out in Table 4. Thus, in an embodiment of the invention, Ergothioneine is provided at least at 0.5 μg/35 g body weight. In a more preferred embodiment, Ergothioneine is provided from about 0.5 μg/35 g body weight to about 5 g/35 g body weight. In a more preferred embodiment, Ergothioneine is provided from at about 1.5 μg/35 g body weight.

In another embodiment of the invention, Vitamin D2 is provided at least at 1 IU/35 g of body weight. In a preferred embodiment of the invention, Vitamin D2 is provided at between 1 IU/35 g body weight to 10 IU/35 g body weight. In a more preferred embodiment, Vitamin D2 is provided at about 2.3 IU/35 g body weight.

In a preferred embodiment of the invention, a product such as ErgoD2™ (see Table 3) is provided at least at 0.5 g/35 g body weight. In a preferred embodiment, a product such as ErgoD2™ is provided at about 1 mg/35 g of body weight.

The various embodiments of the invention, including methods of use or administration of compositions for the treatment of inflammation and oxidative stress or disease states or conditions associated therewith, are useful for a variety of subjects. Mammals may be treated using the methods of the present invention and are typically human subjects. According to additional embodiments, the methods of the present invention may be useful for veterinary purposes with other animal subjects, particularly mammalian subjects including, but not limited to, horses, cows, dogs, rabbits, fowl, sheep, and the like. According to additional embodiments, an animal is any non-human primate, such as for example, a cow, horse, pig, sheep, goat, dog, cat, rodent, fish, shrimp, chicken, and the like.

Methods Involving ETT

As confirmed by research into the significance of the ETT, the presence of the transporter (ETT) indicates the presence and/or need for Ergothioneine (ET). (Gründemann D., The ergothioneine transporter controls and indicates ergothioneine activity—A review, Preventive Medicine (2012), Vol. 54, Supplement 571-574). This reference is incorporated herein by reference in its entirety. Cells lacking ETT are unable to accumulate ET, as a result of the plasma membrane being virtually impermeable for the hydrophilic zwitterion compound of ET. As a result, the existence of the ETT indicates the clear beneficial role for ET as set forth according to the various embodiments of the invention. Immunohistochemistry studies set forth in the Examples of the invention demonstrate that certain cells have strong expression of ETT. According to the methods of the invention, the cells with strong expression of ETT are capable of accumulating ET to higher levels. For conditions disclosed herein, including anemia and diabetes, the accumulation of ET may be critical to treating these disease states and the associated conditions.

According to an embodiment of the invention, the ability to detect the presence, absence, and/or concentration of ETT can be a diagnostic and/or therapeutic method according to the various embodiments of the invention. The diagnostic identification and measurement of the ETT within the membranes of specific cells and/or mitochondria related to various diseases and conditions. Additional description of diagnostic methods is provided in U.S. Application Ser. No. 61/628,162 entitled “Application of the Ergothioneine Transporter SLC22A4 and/or L-Ergothioneine to Targeted Diagnostic Identification and Treatment of Autoimmune Diseases,” which is herein incorporated by reference.

In an aspect of the invention, the absence, presence or specific concentration of ETT, the protein transporter encoded by SLC22A4, in cells may be significant in terms of susceptibility to a particular disease and/or potential to regulate such disease. Polymorphisms of SLC22A4 have been implicated in disease states associated with specific populations, such as rheumatoid arthritis in the Japanese population and with Crohn's disease in a Canadian cohort. (Newman, B. et al., SLC22A4 polymorphisms implicated in rheumatoid arthritis and Crohn's disease are not associated with rheumatoid arthritis in a Canadian Caucasian population, Arthritis and Rheumatism (2005), 52: 425-429). It is an object of the present invention that dosing to particular individuals of ET as part of personalized medicine can lead to modulatory changes in translation of messages from genetic DNA with resultant repair of a disease process.

In a further aspect of the invention, the amino acid L-Ergothioneine, with and without the help of Vitamin D2 has the ability to control and/or modify the transcriptional process. As one skilled in the art shall ascertain, transcription and translation are the steps through which a functional protein is synthesized from the genetic material DNA. These processes are found to occur both in prokaryotes (organisms that lack a cell nucleus or other membrane bound cell organelles) as well as eukaryotes (organisms that have a cell nucleus). Transcription is the first stage of the expression of genes into proteins. In transcription, an mRNA (messenger RNA) intermediate is transcribed from one of the strands of the DNA molecule. The RNA is called messenger RNA because it carries the ‘message’ or genetic information from the DNA to the ribosomes, where the information is used to make proteins. Translation is the process which follows the transcription event. The primary transcript is translated into a sequence of corresponding amino acids forming a peptide chain. These undergo further processing and folding to form the final fully functional proteins. Translation is the process of making peptide strands from primary transcript. There are a set of amino acids which are carried to the site of translation by specific transfer RNAs for the process. Apart from this messenger RNAs and ribosomal RNAs also play significant roles in translation.

The processes of transcription and translation further differ in their regulation. Transcription is highly regulated by internal mechanisms based on chromatin structure, histones, DNA methylation etc. in eukaryotes and operon mechanisms. The operon regulation involves promoter sequences/activators and suppressors which are found in the sequence. Alternatively, translational control is mainly through regulation of binding of ribosomal subunits to the translation complex. Most naturally occurring antibiotics, toxins and drugs target this process. In addition, the post event modifications differ between the processes. Transcriptional product undergoes splicing and dicing events that remove the intragenic portions (introns) which are non-coding in nature. Alternatively, post translational modifications are mainly chemical in nature attaching functional groups to the peptide sequence.

The enzymes involved in transcription and translation further differ as well as the location of the events. A single RNA polymerase is found to be capable of carrying out and controlling the transcription in prokaryotes and three such enzymes are at work in eukaryotes. Alternatively, translation requires several enzymes and factors for the process. It has mainly three steps, initiation, elongation and termination each of which requires a set of RNAs, cofactors and enzymes. Site transcription generally occurs in the nucleus where the transcription factors and enzymes are available. Translation on the other hand occurs in the cytoplasm after the primary mRNA transcript is transferred from the nucleus to the cytoplasm.

The events transcription and translation can be considered as two consecutive processes in production of a functional protein. Both events are controlled by different factors and enzymes but eventually work toward the same goal. Though the regulation, mechanism and other factors differ both are targets for drug designing since they are being controlled by rigorous mechanisms.

It is an embodiment of the present invention, that the amino acid L-Ergothioneine, with and without the help of Vitamin D2 has the ability to control and/or modify the transcriptional process, and/or the translational process, or both.

Paul and Snyder state: “ET protects cellular DNA from damage induced by reactive oxygen species. ETT is abundantly expressed in mitochondria. Mitochondrial DNA (mtDNA) is especially vulnerable to stress, because unlike nuclear DNA, there are no histones to protect it. Mitochondria also lack the very efficient DNA repair mechanisms of the nucleus. The electron transport chain of mitochondria generates free radicals and ROS, such as superoxide and hydroxyl radical that create redox imbalance. In the process, mitochondrial DNA itself is targeted by ROS leading to DNA nicks, breaks and mutations. A region of the mtDNA, the Displacement or D-loop, is a hotspot for DNA damage. Several mutations occurring here are associated with cancers.” (Paul & Snyder, Cell Death and Differentiation (2009), 1-7, Macmillan Publishers Limited).

These scientists further conclude their study provides substantial evidence that ET is a physiologic antioxidant cytoprotectant. ET tissue levels are maintained by its transporter, ETT. Depletion of ETT by RNA interference prevents the antioxidant actions of exogenous ET. More importantly, in the absence of added ET, ETT depletion leads to enhanced oxidative damage of protein, lipid and DNA as well as augmented cell death. In these studies the incubation media contained very low concentrations of ET so that cytoprotection was afforded by ‘endogenous’ ET accumulated by the cells.

Such evidence indicates that ET is a most unusual amino acid with substantial antioxidant efficacy. The existence of a physiologic ET transporter is responsible for high tissue levels. Depletion of ETT leads to augmented oxidative stress and cell death. ET preferentially protects water-soluble proteins from oxidative damage. The high density of ETT in mitochondria implies a unique role in protecting this organelle from the reactive oxygen species that accumulate even with normal oxidative metabolism. ET also protects the cell from damage induced by reactive nitrogen species and UV radiation. For all these reasons ET appears to be an important physiologic cytoprotectant which probably merits designation as a vitamin.

The repair of diseased tissues leads to rapid cell division and differentiation of reactive stem cells. This repair process involves production of toxic byproducts of cellular metabolism, such as cytokines (free radicals). As set forth in the description of the invention, various scientists have promoted the importance of ET in detoxification of these toxic free radicals. It is a further aspect of the invention that ETT is increased in concentration in reactive repair cells to bring needed amounts of ET for neutralization of these toxic free radicals that cause cell death, as described further in Example 4 (study showing control of Paraquat induced oxidative stress/biologic death by ErgoD2™). In a further aspect of the invention, dosing of the medical food ErgoD2™ or foods containing Ergothioneine and enhanced levels of vitamin D2 have the potential to increase the production of ETT within the cellular membranes of reactive stem cells as part of the disease repair process, especially in autoimmune diseases, such as diabetes, rheumatoid arthritis and in certain conditions associated with anemia. As recognized according to the invention, ET tissue levels are maintained by ETT—the transporter, and depletion of ETT by RNA interference prevents the antioxidant actions of exogenous ET.

In a still further aspect of the invention, methods for correcting or modifying genetic polymorphism of the gene with ET dosing may be employed. ETT is highly concentrated in the plasma membrane and mitochondria. Until the recent immunohistochemistry studies performed by us with antibodies for ETT the detailed intracellular localization of ETT was not available for analysis and study. Several facts underscore the role of ET and ETT in DNA protection. The importance of ET is shown by the fact that movement of ET by ETT into cells is a one way mechanism. Once inside, the cell holds on to this important amino acid molecule. In addition, as recognized by Paul & Snyder: “in the absence of added ET, ETT depletion leads to enhanced oxidative damage of protein, lipid and DNA as well as augmented cell death.” (Paul, B. and Snyder, S. H. (2009). “The unusual amino acid L-ergothioneine is a physiologic cytoprotectant, Cell Death and Differentiation.” 1-7). As a result, cytotoxicity and DNA polymorphic changes can be caused by the generation of toxic free radicals such as copper plus 2, iron plus 3, cytokines, etc. The neutralization of these toxic free radicals by ET electron donation can lead to correction of DNA polymorphism through modification of the translation process as previously described.

In a still further aspect of the invention, only cells with strong expression of ETT can accumulate Ergo to high levels. The ability to absorb, distribute. and retain Ergo depends entirely on the specific Ergo transporter (ETT). (Grigat, S., Harlfinger, S., Pal, S. Probing the substrate specificity of the ergothioneine transporter with methimazole, hercynine, and organic cations. Biochem. Pharmacol. (2007), 74: 309-316; Gründemann, D., Harlfinger, S., Golz, S., Discovery of the ergothioneine transporter.” Proc. Natl. Acad. Sci. (2005), 102: 5256-5261; Gründemann, D. The ergothioneine transporter controls and indicates ergothioneine activity—A review. Preventive Medicine (2012), 54: S71-S74). Relevant to the ability of the medical food ErgoD2™ to mobilize iron in diabetic patients (Hermelijn et al., 2013; Evaluation of the Medical Food Ergo-D2® in Diabetic Patients (to be submitted for publication)) are the following published findings. Grundemann states: “The high concentrations of Ergo in mature erythrocytes of all mammalian species examined can now be attributed to expression of a particular carrier. Synchronous uptake of Ergo via ETT and iron via the transferrin receptor might indicate a physiological link” (Gründemann, D., Harlfinger, S., Golz, S., Discovery of the ergothioneine transporter.” Proc. Natl. Acad. Sci. (2005), 102: 5256-5261). Cellular iron uptake and storage are coordinately regulated through a feedback control mechanism mediated at the post-transcriptional level by cytoplasmic factors know as iron-regulatory proteins 1 and 2. This remarkable regulatory mechanism prevents the expansion of a catalytically active intracellular iron pool, while maintaining sufficient concentrations of the metal for metabolic needs (Ponka, P. et al, Function and regulation of transferrin and ferritin, Semin Hematol. 1998, 35(1):35-54).

The following is a summary of the exemplary embodiments of the invention contained herein. Ergothioneine induces anti-inflammatory effects, as demonstrated in an equine inflammatory gum disease model. A preferred embodiment of the invention—filamentous fungi containing Ergothioneine and increased Vitamin D—is effective in reducing or suppressing oxidative stress in a Drosophila model. A preferred embodiment of the invention is effective in decreasing death in in a Drosophila model of Alzheimer's disease, a result not induced by pure Vitamin D. A preferred embodiment of the invention was effective in increasing iron levels in blood in diabetes patients, demonstrating activity of Ergothioneine in increasing iron and blood oxygen carrying capacity by converting toxic ferric iron into soluble, non-toxic ferrous iron. The transporter for Ergothioneine is expressed in a number of tissues, indicating a role for Ergothioneine in a number of conditions and processes: in kidney, playing a role in erythropoietin production; in bone marrow, playing a role in the production of red blood cells; in Langerhans cells of diabetic pancreas, playing a role in production of insulin and/or repair of damaged tissue; arthritic joints and small intestine epithelium of Crohn's disease patients, playing a role in repair of autoimmune disease; in neurons, macrophages and reactive astrocytes in infarcted brain tissue, playing a role in neuroprotective responses in the brain. A preferred embodiment of the invention reduces reduced the severity of disease in a mouse model of Parkinson's disease, indicated by serum IL-6 levels. A preferred embodiment of the invention increased grip and reduced alpha-synuclein levels strength in a mouse model of Parkinson's disease, indicating prevention of development of Parkinson's by antioxidant activity and redistribution of toxic ferric iron to non-toxic, soluble ferrous iron.

All publications and patent applications in this specification are indicative of the level of ordinary skill in the art to which this invention pertains. All publications and patent applications are herein incorporated by reference to the same extent as if each individual publication or patent application was specifically and individually indicated by reference.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Experiments testing the anti-inflammatory effects of mushroom-based formulations with increased natural levels of Ergothioneine according to the invention were tested in an equine inflammatory gum disease study. Elderly horses with inflammatory gum were treated with a mushroom-based formulation, 10 grams per day, for 30-60 days; formulations used were selected because of increased natural levels of Ergothioneine. Horses showed dramatic improvement in the severity of the gum disease within 30-60 days.

### Example 2

### White Blood Cell (WBC) Study

A separate 30 day clinical animal study, involving 36 horses, fed mushroom-based formulations, revealed a statistically significant increase in numbers of white blood cells; mean response among the study sample was 12%. This percentage increase in white blood cells within a 30 day period after dietary supplementation is further supportive evidence for improvement in the animal's immune response and ability to suppress inflammatory diseases, such as gum disease.

Inflammatory disease of the gums is a perfect example of the inflammatory process that occurs in other tissues and/or organ systems, such as arteries, nerves, muscles, heart, colon, and brain, to name a few. The terms inflammation, free radicals, reactive oxygen species (ROS) and oxidative stress are almost interchangeable and a clear understanding of the interactive processes has uncovered new approaches to prevention and amelioration of inflammation and or inflammatory disorders no matter what the origin or location. Similar to the inflammatory processes involved in gum disease, free radicals can perpetuate tissue and organ damage and the disease itself, as well as decrease physiologic function.

The primary function of ET is the protection of RBCs against damage related to ferryl hemoglobin. Monocytes do not express hemoglobin and the role of ET may be another target, such as peroxidases. (Grigat, S. H., Biochem. Pharm., 309-316 (2007); Lagorce J F, Pharmacology, 173-178 (1997)). ET appears to provide protection for monocytes by specific interaction with peroxidase(s). The lack of ET may represent a precipitating factor in the genesis of chronic inflammatory disease (Gründemann, PNAS, 5256-5261 (2005)).

Red Blood Cell (RBC) Study

In the same pilot animal study described above (White Blood Cell Study), the horses also showed a 7.6% mean corpuscular hemoglobin concentration (MCHC) increase within 30 days. RBCs have a 120 day lifespan and we were quite excited by this quick increase in MCHC. The role of free radicals and heme degradation supports the results (Alayash, A., Nat. Rev., 152-159) (2004)).

ET is distinguished from other antioxidants in its interaction with protein-bound heme. No affects are expected on native hemoglobin (HbFeII) by ET, rather ET binds to or react with ferryl hemoglobin (HbFeIV O). The HbFeIV O species is a highly reactive intermediate in the autocatalytic oxidation, caused by many xenobiotics, of HbFeIIO2 to methemoglobin (HbFeIII) and is also considered a starting point for detrimental radical reactions including heme degradation. As supported by the work of Gründemann, the primary function of ET is protecting erythrocytes against damage related to HbFeIV O, demonstrating a role in anemia treatments and prevention.

In addition, this study and a subsequent mouse study confirmed the LD50 is at least 50-100 times the recommended human dose for the compositions according to the invention. As a result, the tested products are considered non-toxic.

Anecdotal reports from people regarding improvements in levels of RBCs, WBCs, and MCHC have filtered in with use of other mushroom-based products, such as ImmuSANO™ and GlucoSANO™ according to additional embodiments of the invention using the compositions described herein.

### Example 3

Pulses of UV radiation of approximately 1-10 J/cm² per pulse, preferably 3-8 J/cm² and most preferably 5-6 J/cm² are used to UV-enhance Vitamin D and/or its derivatives in filamentous fungi. Voltages may also vary based upon safety concerns but should generally be in the range of 1 to 10 or even up to 100 or 10,000 volts as safety mandates. The pulses should generally be in a range of 1-50 pulses per second more preferably 1-30 pulses per second and most preferably 1-10 pulses per second for a range of treatment post-harvest of 0 to 60 seconds.

The inventors used 5.61 J/cm² per pulse on the strobe surface for an input voltage of 3800V and with 3 pulses per second. Sliced mushrooms (Agaricus bisporus, white strain) were placed in the pulsed UV-light sterilization chamber and treated with pulsed light for up to a 20-second treatment at a distance of 17 cm from the UV lamp or 11.2 cm from the window. Control samples did not undergo any pulsed UV treatment. Treated mushrooms were freeze-dried and then sent to a selected commercial laboratory for Vitamin D2 analysis. In this study, a pulsed UV system was also evaluated for effects on the appearance of fresh mushroom slices during a shelf life study.

Results of the experiments demonstrated that pulsed UV-light was very effective in rapidly converting ergosterol to Vitamin D2. Control mushrooms contained 2 ppm dry weight Vitamin D2, while 10 and 20 seconds of exposure to pulsed UV-light resulted in 17 and 26 ppm Vitamin D2, respectively. This increase was equivalent to over 1800% DV Vitamin D in one serving of fresh mushrooms after a 20 second exposure to pulsed UV. The mushrooms treated for 20 seconds also showed no noticeable difference in appearance initially as well as after 10 days of storage at 3° C. compared to the untreated control.

These results compared favorably to the previous pilot study (Feeney, 2006) where mushrooms were exposed to 5 minutes of conventional UV-light exposure. In that study, the mushrooms contained 14 ppm Vitamin D2, but they were also significantly discolored. Hence, the pulsed UV method shows considerable promise as a rapid means to enhance Vitamin D2 levels in fresh mushrooms, theoretically reducing required exposure times from minutes to seconds. Pulsed UV-light exposure did not result in any negative effects on mushroom quality.

Another experiment revealed that pulsed UV-light could rapidly convert ergosterol present in dried oyster mushroom powder to Vitamin D2 (Table 1). These findings indicate that this technology could be used to enrich other mushroom products with Vitamin D2.

TABLE 1
Vitamin D2 generation in dried oyster mushroom powder
exposed to pulsed UV-light (C-type lamp).

| Time of Exposure (s) | Vitamin D2 (PPM) |
|---|---|
| 0 | 8.5 |
| 8 | 15.18 |
| 16 | 24.24 |

The filamentous fungi product is subjected to pulsed UV irradiation after harvest, being irradiated with UV light for a time sufficient to enhance the Vitamin D content thereof. By utilizing UV irradiation, the food product has a substantially increased level of Vitamin D. Preferably, the food product is irradiated with UV radiation, specifically Ultraviolet-B (UV-B), a section of the UV spectrum, with wavelengths between about 280 and 320 nm, or Ultraviolet-C(UV-C), with wavelengths between about 200 and 280 nm. In a more preferred embodiment the UV radiation is pulsed. It is believed that the additional Vitamin D is obtained through the conversion of ergosterol due to the UV irradiation. The time may be the same or increased when the irradiation occurs during the growing process, or post-harvest though the UV irradiation can occur during both periods.

### Example 4

The effect of Agaricus blazei (1-4) on the survival rate of Drosophila melanogaster fed a nutritionally deficient diet, at room temperature (22° C.) was tested using the following parameters: Agaricus blazei (no UV treatment): 1.6 g Vitamin D2/g, dry weight; two pulses of UV-B light: 241.0 g Vitamin D2/g, dry weight; plain yeast paste base as control; vials containing 5.0 ml 1% Agarose medium; yeast paste containing 3% w/w concentration of the two samples.

Drosophila is a model organism with an experimental history of over 100 years. It has a life cycle (embryo to adult) of about 12 days at 22° C. and 9 days at 25° C. The adults live for about 85 days at 22° C. and 60 days at 25° C. under laboratory conditions. It has 3 major chromosomes. Drosophila and human development are homologous processes. They utilize closely related genes working in conserved regulatory networks. Unlike humans, Drosophila can be genetically manipulated. As a result, most of what we know about the molecular basis of animal development has come from studies of model systems such as Drosophila. Drosophila has nearly all the important genes that vertebrates including humans have. Not only the genes are conserved but the pathways regulated by these genes are also conserved. A reliable model using Drosophila as a system to evaluate the effect of a compound for survival on nutritionally deficient diet has been developed by Dr. Krishna Bhat.

The effect of Agaricus Blazei without enrichment, with Vitamin D2 enrichment, pure Vitamin D2 and control (vehicle for the delivery) on the survival rate of Drosophila melanogaster under Paraquat-induced oxidative stress condition was tested. The study focused on the control of Paraquat induced oxidative stress/biologic death. Paraquat is a very potent oxidative stress inducing chemical and causes death in animals and plants by the toxicity of released free radicals. Paraquat (10 mM concentration) (Sigma Aldrich) was used to chemically induce oxidative stress. Paraquat is the trade name for N,N′-dimethyl-4,4′-bipyridinium dichloride, a widely used herbicide. Paraquat, a viologen, is quick-acting and non-selective, killing green plant tissue on contact. It is also toxic to humans when swallowed. This is the most standard chemical used in experimental induction of oxidative stress using the Drosophila model system. It catalyzes the formation of reactive oxygen species (ROS). Paraquat will undergo redox cycling in vivo, gets reduced by an electron donor such NADPH, before being oxidized by an electron receptor such as dioxygen to produce superoxide, a major ROS.

The following materials and methods were utilized. Vials containing 10 mM Paraquat (from Sigma Aldrich) in 5 ml of 1.2% Low melting point Agarose medium were prepared. A strip of half moist filter paper was inserted in the medium (with the wet end in). Yeast paste containing 1% concentration (w/w) of the various test materials (see above) mixed to homogeneity was prepared. Yeast paste without drug was used as control. Uniform aliquot (˜300 mg) of yeast paste with or without the test material) was applied to vials in such a way that yeast paste was on the glass surface and covered the dry end (top) of the filter paper strip. Freshly enclosed wild type isogenized Canton-S males and females were collected and starved on 1% agar medium for 5-6 hours. Four males and females were transferred to the vial containing 10 mM Paraquat in LMP agarose medium and yeast paste with +/−test material (8 flies per vial). 6 vials were used per experiment. Vials with flies were placed horizontally in a tray. The experiment was conducted at 25 degrees C. temperature. Flies were transferred once in 2 days and the number of flies surviving at each transfer was recorded.

Results: Over a period of 10 days, flies fed yeast paste containing A. blazei with Vitamin D2 enrichment showed marked and significant survivability under Paraquat-induced oxidative stress condition compared to the control yeast paste alone (54%+/−10% versus 23%+/−8%), yeast paste containing A. blazei without the Vitamin D2 enrichment (54%+/−10% versus 27%+/−8%), and yeast paste containing pure Vitamin D2 (54%+/−10% versus 13%+/−3%). Vitamin D2 in its pure form had a deleterious effect on the survival and therefore seems to aggravate the oxidative stress.

The results show that a combination of naturally induced Vitamin D2 together with the components of A. blazei has the highest potential and activity to suppress the oxidative stress from Paraquat. These results are highly significant; showing that Vitamin D2, produced naturally by mushrooms, was active only when present within the parent whole food; Vitamin D2 and Vitamin D3 by themselves (i.e. single nutrient or pure Vitamin D2 and Vitamin D3) had no beneficial effect. Oxidative or inflammatory stress was dramatically induced in the Drosophila fruit fly model by the toxic agent, Paraquat, and the end-point of death was evaluated. This model is a very well established paradigm to evaluate oxidative stress. These findings show a novel use for A. blazei enriched with Vitamin D2 for suppressing oxidative stress and associated biologic death.

### Example 5

A. blazei enriched with Vitamin D2 significantly were analyzed to determine whether they enhance the survival and life span of Alzheimer's disease (AD) model in Drosophila. The study evaluated the ability of the edible specialty mushrooms according to the invention, with and without naturally enhanced levels of organic Vitamin D2, to extend the lifespan of the Alzheimer's disease mutant fruit fly.

Type of Model (with specific Drosophila model of neurodegeneration, with references). The targeted over/ectopic expression of APP in the brain using a UAS promoter driven APP transgene, induced by a specific GAL4 trans-driver in the brain of a Drosophila model system, was used for this Example. Below is a reference for such over-expression of APP in the Drosophila model system and the combination gives a fully penetrant AD with limited life-span.

β-Amyloid peptides and amyloid precursor protein (APP) play a deterministic role in Alzheimer's disease (AD). In Drosophila, the targeted expression of the key genes of AD, APP, causes generation of β-amyloid plaques and age-dependent neurodegeneration as well as progression to semi lethality, a shortened life span; genetic manipulations or pharmacological treatments with secretase inhibitors influenced the activity of the APP-processing proteases and modulated the severity of the phenotypes (Greeve I., et al., J. Neuroscience 24, 3899-3906 (2004)). The AD strain lives only for a few days after their eclosion (birth) as opposed to 65 days or more for wild type normal strains.

We determined the extension of life span in the mutant strain for each test compound. We used a specific GAL4 driver that induces the APP gene in the central brain area at high levels (see above) and results in a fully penetrant lethality within a 2-3 weeks period. When these AD flies are given A. blazei enriched with Vitamin D2, the survival rate was increased nearly double that of the control or A. blazei without any enrichment. Treating AD flies with pure Vitamin D2 or Vitamin D3 had no such effect. These results indicate that components in A. blazei, in combination with UV-enriched natural Vitamin D2 has significant benefit against the AD disease.

The results show the ability of a proprietary whole food mushroom with naturally enhanced vitamin D2 to dramatically decrease death by 27%. Statistically significant findings also revealed a physiologic difference between activity of synthetic forms of Vitamin D2 and Vitamin D3 in this neurodegenerative disease model. Study results suggest that neurons may have both Vitamin D2 and Vitamin D3 receptors and that these neuronal cell receptors may be more responsive to Vitamin D2 as compared to Vitamin D3.

### Example 6

The work of Gründemann et al. demonstrates additional sources of ET biosynthesis, including species of cyanobacteria (synthesis confirmed by the detection of the intermediate hercynine). The highest ET content of cyanobacteria in the examined samples was close to 1 mg per g dry mass, which approaches the same level as the top values (1-2 mg per g dry mass) reported previously for several mushrooms. As a result, it is demonstrated that cyanobacteria are a “high density” source of ET.

Previously, the biosynthesis of ET has been demonstrated only in fungi (including edible mushrooms) and mycobacteria, but these are unlikely sources for fishes. In the present study, the origin of ET accumulated in zebra fish was examined. There was virtually no ET, measured by LC-MS, in most tank vegetation (plant, green and red alga). However, ET was detected in a Phormidium sample, a cyanobacterium. In commercial fish feed preparations, ET content increased with the content of cyanobacteria Arthrospira platensis or Arthrospira maxima (Spirulina). High levels of ET (up to 0.8 mg per g dry mass) were measured in cyanobacteria preparations sold as dietary supplements for humans and in fresh Scytonema and Oscillatoria cultures. Thus, cyanobacteria can contain as much as or even more ET than King Oyster mushrooms (Pleurotus eryngii) which we measured at 0.4 mg per g dry mass. All samples with substantial ET content also contained the biosynthesis intermediate hercynine; this strongly suggests that cyanobacteria synthesize ET de novo and can produce high levels of Ergothioneine. Spirulina is a novel, safe, accessible, and affordable source of Ergothioneine for humans.

### Example 7

Human studies have been designed to evaluate the clinical tolerability and potential therapeutic benefits of treating patients with diabetes and anemia with ErgoD2™ Hemo, a 2000 mg ErgoD2™ medical food composition (ergocalciferol (Vitamin D2 11,000 IUs and L-ergothioneine 3 mg). The treatment of patients with ErgoD2™ Hemo provides a composition having naturally high concentrations of the powerful antioxidants L-Ergothioneine and Ergocalciferol (vitamin D2), which work together to naturally elevate red blood cell production and decrease insulin resistance, enabling the body to more easily respond to symptoms experienced by the vast majority of patients taking prescription drugs to treat these conditions.

A human study was conducted to determine the effect of the medical food Ergo D2™ in human diabetic patients, type 1 and type 2. All biomarkers associated with diabetes were evaluated including clinical markers of iron metabolism. As revealed in Table 2, total blood iron levels increased 28% and percentage (%) iron saturation of transferrin increased dramatically by 57%. On the other hand, the total iron binding capacity, which is equivalent to transferrin availability in the blood was reduced by 5-6%.

Diabetes patients very often experience symptoms of hair thinning and loss, or alopecia. In this study, patients receiving treatment with ErgoD2™ according to a preferred embodiment of the invention reported a decrease in arthritic symptoms, an increase in energy in the late afternoon and an increase in hair growth on the head. The attached immunohistochemistry image of a hair follicle (FIG. 1) shows a marked increase in ETT within the stem cells of the hair bulb. The principal detection system for the SLC22A4 (ETT) gene (m-RNA) consisted of a Vector anti-rabbit secondary (BA-1000) and a Vector ABC-AP kit (AK-5000) with a Vector Red substrate kit (SK-5100), which was used to produce a fuchsia-colored deposit. This data further supports the potential activity of ET in stimulating hair growth in the diabetic patients and also its role in increasing iron and blood oxygen carrying capacity to the hair follicle.

TABLE 2
Biomarkers of diabetes in human blood samples following
ergothioneine treatment.

| | Sample | | | |
|---|---|---|---|---|
| Measurement | Size | Difference | % Change | Significance |
| Iron level | 13 | −16.44 ± 7.44 | −28.9% | p = 0.047 |
| Total iron binding | 13 | +17.98 ± 6.50 | −5.53% | p = 0.017 |
| capacity | | | | |
| % iron saturation | 12 | −8.76 ± 3.69 | −57.4% | p = 0.037 |
| | | | (−8.76%) | |
| Mean corpuscular | 14 | −1.20 ± 0.49 | −1.40% | p = 0.030 |
| volume | | | | |

| Measurement | Mean (Pre) | Error | Mean (Post) | Error |
|---|---|---|---|---|
| Iron level | 56.88 | 8.34 | 73.32 | 7.55 |
| Total iron binding | 53.82 | 14.92 | 49.19 | 13.64 |
| capacity | | | | |
| % iron saturation | 15.27 | 2.75 | 24.03 | 3.02 |
| Mean corpuscular | 85.64 | 1.82 | 86.84 | 1.62 |
| volume | | | | |

The data showing mobilization of iron in these diabetic patients is remarkable due to the fact that none of the patients were on iron supplements. It appears that iron is being mobilized from sequestered blocked pools of iron, perhaps through a chelation process and/or that ErgoD2™ is changing toxic Fe+3 to soluble non-toxic Fe+2. Relevant to the ability of the medical food ErgoD2™ to mobilize iron in diabetic patients (Hermelijn et al., 2013) are the following published findings. Grundemann states: “The high concentrations of Ergo in mature erythrocytes of all mammalian species examined can now be attributed to expression of a particular carrier. Synchronous uptake of Ergo via ETT and iron via the transferrin receptor might indicate a physiological link” (Grundemann et al, 2005). Cellular iron uptake and storage are coordinately regulated through a feedback control mechanism mediated at the post-transcriptional level by cytoplasmic factors know as iron-regulatory proteins 1 and 2. This remarkable regulatory mechanism prevents the expansion of a catalytically active intracellular iron pool, while maintaining sufficient concentrations of the metal for metabolic needs (Ponka et al, 1998).

### Example 8

The use of ergothioneine and the compositions of the invention for treatment of anemia, including anemia caused by kidney disease, is supported by scientific evidence that the hormone erythropoietin (EPO) is produced by the kidney, mainly in proximal convoluted tubular cells. When it is produced by the kidney, it travels to the bone marrow and initiates maturation of red blood cells. As a result, without EPO the production of red blood cells is diminished.

Immunohistochemistry studies according to the invention demonstrate the role of ETT and Ergothioneine in bone marrow and the kidney. Antibody titration experiments were conducted with a proprietary rabbit polyclonal antibody to SLC22A4 using steam-based antigen retrieval (pH 6.0 citrate buffer) to establish concentrations that would result in minimal background and maximal detection of signal. Serial dilutions were performed at 20 ug/ml, 10 ug/ml, 5 ug/ml, and 2.5 ug/ml using the antibody on formalin-fixed, paraffin-embedded human tissues supplied by LifeSpan Biosciences and control cell lines (ETTh and CTTh) supplied by Entia Biosciences, Inc. (Dr. Dirk Gründemann) prepared by LifeSpan. The principal detection system consisted of a Vector anti-rabbit secondary (BA-1000) and a Vector ABC-AP kit (AK-5000) with a Vector Red substrate kit (SK-5100), which was used to produce a fuchsia-colored deposit. Tissues were also stained with positive control antibodies (CD31/Vimentin cocktail) to ensure that tissue antigens were preserved and accessible for immunohistochemical analysis. Only tissues that were positive for CD31 and vimentin staining were selected for the remainder of the study. The negative control consisted of performing the entire immunohistochemistry procedure on adjacent sections in the absence of primary antibody. The slides were interpreted by a pathologist and each antibody was evaluated for the presence of specific signal and level of background. Staining was recorded on a 0-4 scale (0=negative, 1=blush, 2=faint, 3=moderate, 4=strong). Slides stained at 2.0 ug/ml were imaged with a DVC 1310C digital camera coupled to a Nikon microscope. Images were stored as TIFF files with Adobe Photoshop.

Using the antibody described in these methods immunohistochemistry results shows the color red/fuchsia indicating the presence of the SLC22A4 gene (i.e. the Ergothioneine Transporter (ETT), which is a peptide). Staining indicates the strong expression of ETT in normal bone marrow of reticulocytes which are the precursor cells for red blood cells. In kidney tissue, namely the proximal convoluted cells (PCT) also show strong staining indicating the presence of ETT. The strong expression in the PCT cells in the kidney demonstrates the cells responsible for producing the hormone EPO.

This activity of the ETT and ergothioneine in erythrocyte progenitor cells and tubular epithelial cells demonstrate that ergothioneine is necessary for the increased production of red cells and erythropoietin. As a result the methods of the invention providing ergothioneine and/or compositions of the invention provide beneficial clinical effects for increasing production of red cells and erythropoietin to treat a patient having anemia.

As confirmed by research into the significance of the ETT, the presence of the transporter (ETT) indicates the presence and/or need for Ergothioneine (ET). (Gründemann, Preventative Medicine, Vol. 54, Supplement 571-574 (May 2012)). As a result, the immunohistochemistry data indicating the presence of the ETT indicates the importance of treatment methods according to the invention, namely to provide Ergothioneine for production of EPO and the production of red blood cells. In addition, the presence of rapidly dividing cells results in some toxic byproducts of metabolism being produced and the methods of the invention, providing Ergothioneine help to neutralize toxic free radicals in order to promote cell survival as opposed to normal metabolism leading to general cell death (i.e. apoptosis).

### Example 9

The use of ergothioneine and the compositions of the invention for treatment of diabetes are also supported by scientific evidence that the production of glucagon and insulin in the pancreas is impacted by the presence of the ETT and therefore ergothioneine. Immunohistochemistry studies according to the invention demonstrate the role of ETT and Ergothioneine in the islets of Langerhans (pancreas cells) (Study done at Lifespan Biosciences, Seattle, Wash.). According to the methods of Example 8, using an antibody that specifically stains the Message from SLC22A4, immunohistochemistry shows the color red/fuchsia indicating the presence of the SLC22A4 gene (i.e. the Ergothioneine Transporter (ETT)).

The faint red-fuchsia staining indicates the faint expression of ETT in normal pancreas tissue, namely the islets of Langerhans responsible for the production of glucagon and insulin. In comparison, ETT is strongly expressed in a pancreas of a diabetic patient. This activity of the ETT and ergothioneine in pancreas cells of a diabetic patient demonstrates that ergothioneine is necessary for the production of glucagon and insulin and/or plays a role in the body's mechanism of repair of these damaged tissues. As a result the methods of the invention providing ergothioneine and/or compositions of the invention provide beneficial clinical effects for improving insulin sensitivity and treating diabetes. A similar activity in the liver could lead to reversal of insulin resistance in that organ as well as in muscle cells and tissues in other parts of the body.

### Example 10

The presence of increased concentrations of the ETT is widely apparent in bodily tissues and/or cells involved in the autoimmune process. This includes for example, rheumatoid arthritis, allergic rhinitis, type 1 diabetes mellitus, Psoriasis, and alopecia areata. By comparison, normal tissues and diseased non-autoimmune tissues showed much less presence of the ETT. The potential reparative activity of Ergothioneine in autoimmune diseases, including type 1 and type 2 diabetes, is supported by examples in normal joints and rheumatoid arthritis joints using the methods disclosed in Example 8. Moreover, many diabetic patients have joint pain associated with increased joint inflammatory changes. Please see clinical response in Example 7.

Synoviocytes and subsynovial histiocytes of a normal, healthy joint show negative to faint staining, indicting the lack of ETT in the tissues. The vascular smooth muscle was faintly positive, whereas fibroblasts were negative. In comparison, synoviocytes and subsynovial histiocytes of a patent having rheumatoid arthritis show moderate to patchy focal strong staining in the tissue, indicting the increased presence of ETT in the tissues. In addition, reactive capillaries were moderately positive; infiltrating macrophages were strongly positive; plasma cells were moderate to strong; lymphocytes were faint. The rheumatoid arthritis sample showed significantly increased staining of reactive synoviocytes and subsynovial histiocytes, and strong staining of infiltrating macrophages, as well as increased staining of reactive fibroblasts and capillaries.

### Example 11

The presence of increased concentrations of the ETT is widely apparent in bodily tissues and/or cells involved in the autoimmune process. This finding further includes Crohn's disease. By comparison, normal tissues and diseased non-autoimmune tissues showed much less presence of the ETT. The potential reparative activity of Ergothioneine in autoimmune diseases, including type 1 and type 2 diabetes, is supported by examples in patient's having Crohn's disease using the methods disclosed in Example 8.

Sections of a normal, healthy small intestine show faint staining. The absorptive epithelium was faintly to moderately positive, and goblet cells showed faint staining. Plasma cells within the lamina propria showed moderate staining, and macrophages were moderate. Vessels within the submucosa showed faint to moderate staining of endothelium and faint staining of smooth muscle. Within enteric ganglia, ganglion cells were faint to moderate and Schwann cells were blush to faint. Smooth muscle of the muscularis mucosa and muscularis propria were blush positive, and fibroblasts were faint.

In comparison, small intestine show changes consistent with Crohn's disease. Reparative epithelium and epithelium deeper in crypts showed variable faint to moderate staining. Plasma cells in the lamina propria were positive. Collections of histiocytes were moderately positive. In areas of ulceration, collections of macrophages and plasma cells were moderately to strongly positive. Lymphocytes were mostly negative. In areas of inflammation, reactive capillaries showed moderate staining of endothelial cells. Muscular vessels within the submucosa showed faint staining within endothelial cells and vascular smooth muscle. Within enteric ganglia, ganglion cells were faint to moderate and Schwann cells were blush to faint. Compared to normal colon, samples showing inflammation consistent with Crohn's disease showed increased staining of plasma cells and macrophages in areas of inflammation and ulceration, with increased staining of reactive capillaries.

### Example 12

As suggested by Rogers et al. in Example 13, the etiology of stress, and in this case the stress events of Parkinson's disease, IL-6 has been shown to generate neuroprotective responses to brain neurons after stroke (2).

IL-6 is an antioxidant biomarker and an active peptide. In an aspect of the invention this biomarker may be related to ERGO (ET) and/or ETT, such as the two are working in conjunction and/or functioning as a signaling marker. Applicants' research provides confirmatory evidence as shown in Sample 2 and 3 of FIG. 2. (Ponka, P. et al, Function and regulation of transferrin and ferritin, Semin Hematol. 1998, 35(1):35-54).

### Example 13

### Antioxidant Diets and Interleukin-6 Levels in the Plasma of Parkinson's Mice

Overview.

Interleukin-6 has long been recognized as the blood cytokine that mediates the transcription of the protective acute phase reactant proteins made in the liver after acute stress and/or trauma (1). Neuroimmune mechanisms conversely may be involved in the neurodegenerative process of Parkinson's disease (PD), inclusive of the role of LRRK as genetic marker of familial PD although the details of both toxic and protective pathways require further examination. Pertinent to the future therapies based on the etiology of stress, and in this case the stress events of Parkinson's disease, IL-6 has been shown to generate neuroprotective responses to brain neurons after stroke (2). There is a role of chronic infection and inflammation (including IL-6) in the gastrointestinal tract in the etiology and pathogenesis of idiopathic Parkinson's disease (3). The intestinal dysfunction appears to be muscular in origin. Further supporting muscular problems in PD are the findings that PD patients exhibit motor incoordination and loss of grip strength.

As a biomarker, the level of interleukin-6 in cerebrospinal fluid inversely correlates to severity of Parkinson's disease. (4). Serum levels of IL-6 were also evaluated in clinical cohorts of PD patients ((5). Here, IL-6 levels were found to be similar between PD patients (treated and not treated) and controls. However, there was a negative correlation of IL-6 levels and the activities of daily living scale (P<0.05), indicating that patients with more severe disease have higher levels of this cytokine.

We pursued this effect in our mice since the role of IL-6 and other neuroimmune factors need to be elucidated on PD. Alpha synuclein transgenic mice expressing the human gene for alpha-synuclein were treated with the medical food ErgoD2™ (Pleurotus; Table 3) or N-Acetylcysteine (NAC), and whole blood cell lysate samples from these mice were evaluated in triplicate using an ELISA for the presence of IL-6. The results were compared to those from untreated and wild-type controls.

TABLE 3
Product information for exemplary embodiment of the
invention ErgoD2 ™ (Pleurotus)

| | |
|---|---|
| Product | USDA Certified Organic |
| Vitamin D2 | 5,000 IU/gram |
| Ergothioneine | 1.5 mg/gram |
| Product Code | EPE-200 |
| Source | Whole Fruit Body |
| Country of Origin | United States of America |

Nutritional Profile

| | | | |
|---|---|---|---|
| Protein (% total | 25.0% | Calories | 3.46/g |
| weight) | | | |
| Carbohydrate (% | 56.6% | Sodium | 0.36 mg/g |
| total weight) | | | |
| Fat | 2.2% | Fiber | 32.7 g/100 g |
| Ash | 8.2% | Cholesterol | 0 |
| Moisture | 8.0% | Trans-fat | 0 |

Properties

Controls inflammation associated with oxidative stress in various organ
systems. Nutritionally supports the immune system and balances cellular
function. Antioxidant, antitumor, antifungal, antiarthritic, antiviral,
powerful anti-inflammatory.

Application

| | |
|---|---|
| Biopharmaceutical | Control of inflammation damage in the liver, |
| | kidneys, and brain. Regulates blood sugar and |
| | immune system. Promotes healthy circulation, |
| | cellular metabolism and function. |
| Supplementation | Value-added ingredient. Rich source of potassium, |
| | protein, and selenium along with the nutrient rich |
| | polyphenols and Beta-glucans. |
| Food | Natural preservative, value-added ingredient, |
| | umami sensory properties. |
| Cosmetics | Value-added ingredient in nutricosmetics and |
| | cosmeceuticals. Exfoliating qualities for use in |
| | scrubs and skin rejuvenation. |

Results.

A serial dilution of provided standard solution allowed for the creation of a standard curve following absorbency reading. An exponential regression of this curve was used to extrapolate the concentration of IL-6 in the lysate samples based on their final absorbency values and accounting for dilution factors of the samples.

Lysate samples taken from subjects #222 and #321 were clotted. A small amount of each was used during assay, but their relatively high corresponding dilution factors call into question the findings based on these samples. The uncertainty of these data is reflected in the corresponding standard error of the mean for each sample.

For the remaining lysate sample from the ErgoD2™ (Pleurotus)-treated group (ID#221), a greater amount of IL-6 was detected by assay compared to the detected amounts from the untreated and wild-type groups (FIG. 3C; p=0.002, and p=0.0005, respectively). The ErgoD2™ (Pleurotus)-treated subject expressed 16.9 pg/mL more IL-6 than did the untreated group, and 17.7 pg/mL more than did the wild-type group.

For the remaining lysate sample from the NAC-treated group (ID#322), a greater amount of IL-6 was detected by assay compared to the detected amounts from the untreated and wild-type groups (FIG. 3D; p=0.02, and p=0.02, respectively). The NAC-treated subject expressed 8.7 pg/mL more IL-6 than did the untreated group, and 9.5 pg/mL more than did the wild-type group.

There was no statistically significant difference in the measured expression of IL-6 between the untreated and wild-type mouse groups.

Conclusion.

Treatment with ErgoD2™ (Pleurotus) or N-Acetylcysteine (NAC) increases the presence of blood IL-6, compared to untreated and wild-type control groups. Lysate samples taken from whole blood cells from transgenic mice expressing the human gene for alpha-synuclein were tested via ELISA protocol. ErgoD2™ (Pleurotus) treatment group samples exhibited 16.9 and 17.7 pg/mL more IL-6 concentration than did untreated and wild-type group samples, respectively. NAC treatment group samples exhibited 8.7 and 9.5 pg/mL more than did untreated and wild-type group samples, respectively.

Additional assays using more subjects in each treatment group will better elucidate the actual change in IL-6 concentration in response to each treatment paradigm and how this response may vary across different subjects.

There was a trend towards statistically significant increase in IL-6 concentration between the untreated and wild-type groups. This suggests that IL-6 concentration may increase in response to ergothioneine based therapies to offset pro-oxidant targeting to generate this abnormal protein expression as occurs in neurodegenerative disease conditions and also from acute and chronic stress. The reason and mechanism by which this occurs should be further investigated.

The relevance is that IL-6 can induce an adaptive ‘acute phase response’ to help the body better cope with stress. It seems the ergo and NAC antioxidants may be priming this protective system in our mice.

A further potential application of these findings is in the treatment of muscular dysfunction in both normal and disease conditions. The elevation of key protein signaling antioxidant molecules, IL6, glutathione, and alpha synuclein also occurred in those animals that experienced increased grip strength and improvements in motor coordination. (See Rogers J T. Ferritin translation by interleukin-1 and interleukin-6: the role of sequences upstream of the start codons of the heavy and light subunit genes. Blood. 1996 87(6): 2525-253).

### Example 14

A study at Harvard Medical School/Massachusetts General Hospital, Boston, Mass. studied the effect of the medical food ErgoD2™ on grip strength and alpha synuclein levels in a Parkinson's disease mouse model.

Study Methods and Materials

Mouse Cohorts:

Matched cohorts of an SNCA transgenic mouse model of PD were used to measure the therapeutic efficacy of the medical food formulation ErgoD2™, created from a single mushroom species, Pleurotus eryngyii. The outcomes measurements assessed improvement in grip strength and alpha-synuclein levels in the cortex and/or midbrain of the PD animals. As outlined in procurement form Jackson labs, PAC-Tg (SNCAWT); Snca−/− mice do not show any enteric nervous system abnormalities or widespread α-synuclein aggregation in brain or colon. No detectable motor behavior impairments, autonomic abnormalities, olfactory dysfunction, dopaminergic deficits, Lewy body inclusions or neurodegeneration are associated with α-synuclein expression in these mice (Kuo et al., 2010). As outlined in the results section, the mice were grouped at ten (10) mice/cohort and were fed a purina diet for three months with and without addition of the medical food ErgoD2™, provided by Entia Biosciences, Inc.

Diet:

Three (3) test diets were created with the assistance of Carrie Schultz, Ph.D., Land O'Lakes Purina Feed, and each diet was color-coded. The control/placebo mouse diet consisted of Purina Modified LabDiet® (RMH 3000). The two treatment diets were RMH 3000 supplemented with (i) synthetic N-Acetyl Cysteine (NAC) @ 0.015% of total weight and (ii) the medical food ErgoD2™ @ 0.5% of total weight. All diets were manufactured by the Purina Animal Nutrition facility in Richmond, Indiana and maintained at the recommended storage conditions of −4 degrees to 4 degrees centigrade and 50% or less relative humidity.

ErgoD2™ is a 100% USDA certified organic formulation created from a single mushroom species, Pleurotus eryngyii, which contains naturally high concentrations of L-Ergothioneine (Ergo) and enhanced levels of Ergocalciferol (vitamin D2) using proprietary UV light enhancement technology from Entia Biosciences, Inc. The amount of ErgoD2™ added to the RMH 3000 was based on the recommended daily human dosage equivalent ratios (Table 4).

TABLE 4
Dosage of a preferred embodiment of the invention in mice and humans.

| | Body | ErgoD2 ® | | |
|---|---|---|---|---|
| | Weight | Dosage | Ergo | D2 |
| | Grams | MGs | MGs | IUs |
| AVG Mouse | 30 | 0.85714 | 0.00129 | 1.97143 |
| AVG Human | 70,000 | 2,000 | 3 | 4,600 |

Brain and Blood Collection:

Each group of animals was sacrificed and their brain cortices and mid-brain sections retrieved and stored at −70° C. Cortex and midbrain samples were manually homogenized separately in 1 mL of RIPA buffer (Boston Bioproducts) supplemented with protease inhibitors. The samples were then centrifuged at 4° C. for 15 minutes, and the supernatant aliquoted and stored at −70° C. Whole blood samples were centrifuged at −10° C. and the supernatant (plasma) collected and stored at −70° C.

Alpha-synuclein was measured using an ELISA kit (Invitrogen, Hu α-Synuclein ELISA Kit) according to the manufacturer's protocol. The α-synuclein concentration of each 1 mL homogenized midbrain lysate was calculated by interpolating absorbance readings to a standard curve and adjusting for necessary dilution factors and protein equivalencies. The mean α-synuclein concentration of the samples in each treatment group was then calculated. Treatment groups were further divided into male and female subjects for additional analysis. An analysis of variance demonstrated a statistically significant difference between the treatment groups (p<0.005). Post-hoc analysis using a one-tailed Dunnett's t-test [H(i): μ(i)<μ(untreated)] revealed that both the ErgoD2™ and NAC treatment groups expressed lower amounts of α-synuclein than did the untreated group (p=0.001 and p=0.027, respectively). The ErgoD2™ treatment group expressed about 22% less α-synuclein, and the NAC treatment group about 14% less α-synuclein, than did the untreated group.

Grip Strength:

To assess the effect of dietary antioxidant formulation on neuromuscular function and muscular strength in PD mice, a grip strength task was performed using Chatillon Ametek Digital Force Gauge, DFIS 2, (Columbus Instruments) after three months of formulation treatment. A mouse was raised toward a triangular metal transducer and allowed to grab onto a transducer with its forelimbs. The mouse is then pulled back gently until the grip is released and peak force at the point of release is recorded in Kg. Three consecutive trials were performed for each mouse and the average used for statistical analysis.

Data Analysis and Statistical Analysis

Bicinchoninic acid assays were performed on all plasma and brain tissue samples to calculate equivalent protein concentrations for use with additional bioassays. Plasma, midbrain and cortex samples were tested using a commercially available enzyme-linked immunosorbent assay for alpha-synuclein (Invitrogen, Camarillo, Calif., USA).

Analysis of variance was used to assess the differences between treatment groups in subject grip test performance and brain and blood sample alpha-synuclein concentrations. For each study, post-hoc multiple pairwise comparisons were performed after confirmation of homogenous group variance using Levene's test of equality of error variances. One-tailed Dunnett t-tests were used for post-hoc comparisons if corroborated by a directional hypothesis a priori.

Relationships between significant data measures were evaluated using Pearson and partial linear and logarithmic correlation metrics. A multinomial logistic regression model was used to examine the trend strength with which these relationships varied between treatment groups.

Results Results from the grip test and α-synuclein bioassays were tabulated for additional analysis (see FIG. 4A). An analysis of variance revealed that grip test performance improved by 10.9% for all ErgoD2™ treated subjects vs. untreated controls (FIG. 4B; p=0.044, 135.23±10.14 vs. 121.97±13.69 kg).

Plasma and cortex alpha-synuclein concentrations were comparable with no significant difference between each treatment group and untreated controls. However, midbrain alpha-synuclein levels were 21.9% and 14.0% lower in ErgoD2™ (p=0.001) and NAC (p=0.027) treatment group tissue samples, respectively, than in untreated controls (FIG. 4C; 1.97±0.42 and 2.17±0.31, vs. 2.52±0.28 μg/mL).

Relationships between the two significant findings—grip test strength and midbrain α-synuclein concentration—were assessed using a Pearson partial correlation of their logarithmic forms while controlling for the sex of the subject mice. A negative correlation trending toward significance was noted (FIG. 5A; r=−0.275, p=0.075).

A multinomial logistic regression was used to better model the relationship between grip test strength and midbrain α-synuclein concentration while accounting for the possible different efficacies of each treatment (see FIG. 5B). This model more clearly demonstrated an inverse correlation between the two measures, whereby higher (lower) α-synuclein levels predicted lower (higher) grip test strengths for each treatment group compared to untreated controls (χ²(6)=24.968, p=0.000). It should be noted that both a decrease in midbrain α-synuclein and increase in grip strength was predictive of treatment with ErgoD2™ whereas NAC treatment was significantly correlated only with decreased α-synuclein. The logistic model demonstrated high classification accuracy for all three treatment groups (ErgoD2™: 60%, NAC: 60%, untreated: 80%).

Discussion and Conclusion

There is compelling support for the concept of limiting expression of alpha-synuclein (SNCA) in the brains of Parkinson's disease (PD) patients. Duplication or triplication of the SNCA gene can lead to familial PD where increased levels of this pathogenic protein is correlated to severity of symptoms (i.e., triple repeat SNCA patients show dementia) (Ahn et al., 2008; Chartier-Harlin et al., 2004; Ikeuchi et al., 2008; Singleton and Gwinn-Hardy, 2004). Polymorphic variability in the promoter of the SNCA gene was linked to its increased expression as a risk factor for PD (Chiba-Falek et al., 2006). Experimental siRNA infusions demonstrated that it is possible to limit SNCA in vivo as a therapy for PD (Lewis et al., 2008).

In this study, we demonstrate for the first time that a natural mushroom-based medical food ErgoD2™, containing potent antioxidant bionutrients, can reduce midbrain α-synuclein expression correlated to improved grip performance in a wild type α-synuclein transgenic model—PAC/SNCA mouse. These mice provide a clear model for wild type α-synuclein over-expression including impaired grip strength, and as such, represent an excellent model for non-genetic late onset forms of Parkinson's disease.

It is well established that oxidative stress plays an important role in the pathogenesis of PD, triggering a physiological cascade that includes mitochondrial dysfunction, striatal dopamine loss, inflammation, neurodegeneration, and, potentially, accumulation of α-synuclein protein ((Olivares et al., 2009), (Rogers et al., 2011), (Mikkilineni, 2012), (Ross et al., 2011). Though the exact relationship between oxidative stress and α-synuclein over-expression in PD is not yet fully understood, it is clear that the two phenomena are intimately related and each critical to the development of PD's signs and symptoms.

There is little question but that oxidative stress plays a role in the onset and progression of PD. It is also known that increases in iron levels, via Fenton chemistry, is a very potent catalysis of oxidant damage (Hahl et al., 2013). An additional culprit in PD pathogenesis is the accumulation of Nigral iron. (Kaur et al., 2003). We recently reported in Cell (Duce et al., 2010) and Nature Medicine(Lei et al., 2012) the participation of major neurodegenerative disease proteins, APP and tau, in iron homeostasis.

We have identified well-tolerated FDA drugs, including Phenserine, an acetylcholinesterase (AChE) inhibitor used in treatment of Alzheimer's disease (AD) that selectively blocked SNCA mRNA translation by targeting the 5′untranslated region (5′UTR) (FIG. 1). Our laboratory is now reporting that the SNCA 5′UTR is a uniquely modified iron-responsive element (IRE) RNA stem loop, which binds to iron-regulatory protein-1 (IRP1) to control iron-dependent SNCA translation. The SNCA IRE controls translation differently from the classic IRE stem loop of H-ferritin mRNA (iron (Fe) storage), which interacts with both IRP1 & IRP2 as translation repressors (Rogers et al., 2008; Thomson et al., 2005) (Wang et al., 2007).

The anemia or low hemoglobin levels preceding Parkinson's disease in a case-control study (Savica et al., 2009) and the profile of α-synuclein gene expression tracks with the profile of genes closely linked to erythropoeisis and iron usage in the synthesis of hemoglobin (Scherzer et al., 2008).

In the separate clinical diabetes study of Example 7, using the same medical food, ErgoD2™, but at a higher human dose, there was a significant increase in blood iron and total percentage iron saturation, despite the fact that no patients received iron supplements (Table 2). In essence, there is a potential picture that the results seen in the ErgoD2™ PD mouse study, may be related to redistribution of iron within iron functional pools in tissues and organs systems, such as the brain. Thus, we present a model that potent antioxidants within ErgoD2™ can mobilize and redistribute iron to the blood with resultant erythropoiesis. It has also been shown that SNCA and the heme metabolism genes ALAS2, FECH, and BLVRB form a block of tightly correlated gene expression in 113 samples of human blood (Scherzer et al., 2008).

This redistribution of iron from the brain may cause inactivation and repression of the SNCA mRNA IRE in the mid-brain and consequent improvement of muscle function as measured by grip strength. As an additional positive indicator of these events, we observed that blood α-synuclein levels were increased, potentially iron responsive, which is consistent with the report that α-synuclein is co-expressed with the heme synthetic pathway of ferrochelatase and erythroluvinenyl synthase (eALAS) (Scherzer et al., 2008).

In the human diabetes trial of ErgoD2™ currently in progress (Hermelijn, 2013), initial results in (Table 2) show a redistribution of iron within the blood of patients; total blood iron levels and total percentage iron saturation were increased after 90 days of use. We therefore, in our SNCA mouse model for α-synuclein over-expression hypothesize that modest antioxidant metal chelators, such as ErgoD2™ and perhaps NAC, may sequester iron from the brain Substantia nigra to reduce toxic fibrillated α-synuclein and thereby alleviate PD motor problems such as impaired grip strength. A major mechanistic axis is that iron chelation with ErgoD2™ may well cause Iron-regulatory protein suppression via α-synuclein via its 5′UTR specific iron-responsive element. Thus, ErgoD2™ may well suppress α-synuclein translation via IRP1 in an axis that lessens dopaminergic neuronal lesions in a non-toxic manner and thereby alleviate motor symptoms of PD, as shown by the increase in grip strength.

A recent meta-analysis assessing studies involving more than 130,000 people in Europe, North America, and Australia suggested that disrupted iron metabolism may be an important factor in the pathogenesis of Parkinson's disease. Their study also suggests a causal association between increased serum iron levels and decreased risk of developing Parkinson's disease (Pichler, I. et al., Serum iron levels and the risk of Parkinson's disease: A mendelian randomization study. Plos Med., June 4, Journal Pmed. 1001462, published online June 4).

Antioxidants and Parkinson's Disease:

Recent studies have demonstrated marked reductions in total glutathione in the substantia nigra of the Parkinson's brain preceding the onset of other pathological indicators including neurodegeneration and α-synuclein over-expression. This finding suggests either a cause-and-effect relationship between oxidative stress and neuronal α-synuclein accumulation or a shared underlying etiology. Regardless, increased α-synuclein expression in the midbrains of Parkinson's patients has proven a reliable predictor of both disease progression and symptom severity. Therefore, quantification of α-synuclein expression in the midbrain following any targeted treatment protocol, especially the antioxidant treatment protocol in this study, represents a valid method of assessing treatment efficacy.

In this study, the medical food ErgoD2™ containing known potent antioxidant activity was assessed for potential efficacy in treating Parkinson's disease by measuring post-mortem midbrain α-synuclein expression in treated and untreated Parkinson's model mice. Midbrain samples were tested exclusively after an earlier pilot study suggested that therapeutic indicators were more likely to occur in the midbrain than in the cortex. This approach aligns with theory that places primacy on neurodegeneration and α-synuclein accumulation in the substantia nigra of the Parkinson's brain. Data analysis revealed that both ErgoD2™ and the control antioxidant N-Acetylcysteine (NAC) demonstrated efficacy in lowering α-synuclein levels in the midbrains of Parkinson's model mice; ErgoD2™ had a significant greater reduction. Though a pilot study suggested a possible stronger benefit for female mice, the present study, using larger sample sizes and greater statistical rigor, did not duplicate this finding. In any case, these results certainly warrant additional validation and further testing to elucidate the exact therapeutic potential of these antioxidant compounds and if neurochemical changes can be connected to real symptomatic improvements.

Grip Test and Parkinson's Disease:

Grip testing conducted on the PD model mice treated with ErgoD2™ and the control NAC also demonstrated statistically significant improvement. Accounting for the sex of the mice revealed that female mice showed the most marked statistically significant improvement.

Other Trends of Note:

The test results themselves were different for male and female mice. This finding indicates the need to subdivide treatment groups by sex, preferably with equal numbers of mice in each subgroup. Likewise, the relatively small sample size in each mouse population after accounting for and separating by gender calls for extending the analysis to larger matched groups.

ErgoD2™ treated mice, especially females, showed statistical significant improvement in Grip Test. This result may parallel differences seen in human PD symptomatology and clinical manifestations between females and males (Simunovic, 2010). Moreover, these gender differences could correlate with symptom response and changes in α-synuclein midbrain levels. The increases in grip strength in this PD study may be related to the enhanced levels of natural Vitamin D2 present in the ErgoD2™ medical food. A recent orthopedic study revealed that maintaining Vitamin D levels—as measured by 25(OH)D levels—helped diminish muscular weakness after intense exercise (Barker, 2013).

There is a distinct possibility that the medical food ErgoD2™ mobilizes iron from sequestered pools with resultant reduction in fibrillated toxic α-synuclein and alleviation of PD motor symptoms. The findings of increased blood iron and total percentage iron saturation in the human diabetes study, despite no iron supplements, is further evidence for the iron chelating powers of ErgoD2™. It is entirely logical that toxic iron sequestration may be a problem in other neurodegenerative diseases, such as Alzheimer's disease, multiple sclerosis and autism. This apparent ability of the ErgoD2™ medical food to mobilize and enhance iron metabolism in the brain warrants further clinical studies.

These results showing increased grip strength and increased blood iron levels, which allows better oxygen carrying capacity support our claim regarding the physiologic ability of Ergothioneine and Vitamin D2 to increase athletic performance.

### Example 15

### Glutathione Levels are Increased in Human Alpha-Synuclein Transgenic Mice in Response to Antioxidant Diets Especially Those Mice Fed the Pleurotus Diet, ErgoD2™

Oxidative stress has been associated with the etiology of Parkinson's disease (PD) (Ben-Shachar and Youdim, 1990; Martin et al., 2012). For example, the familial PD-associated gene product DJ-1 (Park-7) up-regulates glutathione synthesis during oxidative stress and inhibits A53T alpha-synuclein toxicity (Zhou and Freed, 2005). An early biochemical change seen in PD is a reduction in the levels of total glutathione, as key cellular antioxidant. Here randomized, double-blind, pilot evaluations were conducted for levels of intravenous glutathione in Parkinson's disease (Hauser et al., 2009). Traditionally, it has been thought that this decrease in GSH levels is the consequence of increased oxidative stress, a process heavily implicated in PD pathogenesis (Canals et al., 2001). However, emerging evidence suggests that GSH depletion may itself play an active role in PD pathogenesis (Martin and Tesmann, 2009).

Our Pac-Tg (SNCA) mice transgenic mice express the human gene for human alpha-synuclein and these were assigned one of four treatment paradigms (including no treatment). Plasma samples taken from these mice were evaluated in duplicate using a kinetic assay protocol for the presence of glutathione (GSH), a measure of resilience to oxidative stress. The tested subject groups were treated with one of the following: Blazei Mushroom, Pleurotus mushroom, or n-acetyl cysteine (NAC). The results for these groups were compared to those for both untreated and wild-type controls.

Data. A serial dilution of provided standard solution allowed for the creation of a standard curve following kinetic absorbance readings (FIGS. 6A and B). The change in absorbance (Vmax) over five minutes (six absorbance readings) was calculated for each standard and plotted against each standard's known GSH concentration. Readings for the first (and most concentrated) standard were omitted because they did not demonstrate the anticipated increase in absorbance with time. The change in absorbance for the second standard was calculated using only the first two kinetic readings (t=0, t=1) for the same reason. Working with the data in this way, however, ultimately allowed for the creation of the anticipated upward-sloping, linear standard curve. A linear regression of the resulting standard curve allowed for extrapolation of GSH levels in the assayed plasma samples based on their changes in absorbance (Vmax) over six minute-reads.

Following tabulation of the data, results were omitted if they were not reproducible in duplicate using all six kinetic readings and within 30% precision. In order to conduct reasonable statistical analysis, one additional wild-type was included for which the fifth and sixth kinetic reads were omitted. Even though fewer reads were used to calculate the Vmax of this sample, an expectedly upward-sloping, linear change in absorbance was evident (FIG. 6C).

Analysis.

All sample data that did not produce an upward-sloping, linear change in absorbance using all six minute-reads in reasonable duplicate were assumed to contain protein flocculant, and were discarded from analysis (except in the case of the previously-mentioned wild-type subject).

An analysis of variance of the remaining data demonstrated that there was a statistically significant difference in GSH concentration between the plasma samples in each treatment group (p=0.001). A Fisher post-hoc analysis revealed that the plasma samples taken from the wild-type group contained higher concentrations of glutathione than did samples taken from any other group: from each the Blazei-treated group (p<0.001), the Pleurotus-treated group (p=0.001), the NAC-treated group (p=0.001), and the untreated group (p<0.001) (FIG. 7C).

This difference in plasma GSH concentration reflects the well-established observation that glutathione levels decrease in response to oxidative stress, as has been associated in PD (Canals et al., 2001). Plasma samples from wild-type, non-transgenic mice contained significantly higher concentrations of glutathione than did those from Parkinson's-model, transgenic mice expressing the human alpha-synuclein gene as would be predicted form clinical correlated data in humans((Scherzer et al., 2007). The oxidative stress experienced by the Parkinson's model mice is reflected by lower plasma GSH concentrations.

Continued post-hoc analysis could establish a potentially statistically significant difference between the transgenic treatment groups. Two results that trended toward statistical significance were noted: that plasma samples from each Pleurotus-treated mice and NAC-treated mice contained higher concentrations of glutathione than did those from untreated mice (FIG. 7D; p=0.10 and p=0.09, respectively). While frank statistical significance can be established between the Pleurotus or NAC-treated group and untreated group to the p values shown—even using the fairly liberal analytic criteria implemented here—the trending increase in glutathione concentration with Pleurotus or NAC treatment reflects the hypothesized therapeutic, antioxidant properties of these compounds.

Because of the limited data-set, an analysis that accounted for the sex of each subject mouse in each treatment group could not be performed with an appropriate level of statistical confidence. A preliminary examination of the data when accounting for sex, however, could indicate at most (and using beyond the very most liberal statistical criteria) potentially higher plasma glutathione concentrations in male mice treated with Pleurotus mushroom than in females or untreated controls (FIG. 8). Further analysis with greater numbers of samples would have to be conducted to elucidate the size or legitimacy of this possible effect.

Conclusion. A kinetic assay was used to measure plasma glutathione concentrations in transgenic mice expressing the human gene for alpha-synuclein and treated with either Blazei mushroom, Pleurotus mushroom, or N-acetylcysteine, and the results were compared to sample concentrations from untreated and wild-type controls.

Plasma glutathione (GSH) is indicative of resilience to oxidative stress, just as a decrease in plasma glutathione indicates the presence of oxidative stress. Reduced glutathione levels have been associated with amyloidopathies including Parkinson's and Alzheimer's disease, reflecting the presence of oxidative stress in these conditions.

It is no surprise, then, that the tested wild-type mice expressed higher levels of plasma glutathione: these mice were not modified to model Parkinson's disease and therefore were not experiencing associated oxidative stress. By contrast, all transgenic Parkinson's model mice expressed significantly lower levels of plasma glutathione, suggesting the presence of oxidative stress in these subjects.

If decreased GSH is indicative of oxidative stress and this oxidative stress is responsible for the physiological decline associated with Parkinson's disease, then treatments that decrease oxidative stress should ameliorate Parkinson's symptoms. It's reasonable to assume, then, that increased levels of glutathione following treatment reflect lower levels of oxidative stress and a therapeutic effect of treatment. Though such a finding could not absolutely be established by the present study, both Pleurotus and N-acetylcysteine treatment trended toward a statistically significant increase plasma glutathione concentration compared to untreated controls. These findings suggest a possible role for Pleurotus mushroom and N-acetylcysteine, two compounds with known antioxidant properties, in the treatment of Parkinson's disease.

1: A method of improving athletic performance, muscle function and reducing injuries in mammals comprising:
administering to said mammal in need thereof a source of Ergothioneine and/or Vitamin D; and
obtaining physiological improvement or preventing or repairing an injury in said mammal.

2: The method of claim 1 wherein said physiological improvement comprises strength, endurance, balance, or conditioning.

3: The method of claim 1 wherein said mammal has a disease with neuromuscular or muscular dysfunction selected from the group consisting of Parkinson's disease, multiple sclerosis, and Guillan-Barre syndrome.

4. (canceled)

5: The method of claim 1 wherein said source of Ergothioneine and/or Vitamin D comprises a whole food and/or Cyanobacteria source and/or is a naturally enhanced, filamentous fungi, tissue, substrate, spent substrate or component thereof, with increased levels of Vitamin D.

6. (canceled)

7: The method of claim 1 wherein said Vitamin D is Vitamin D₂.

8: The method of claim 5 wherein said filamentous fungi is a mushroom species selected from the group consisting of: Pleuorotus eryngii, Agaricus bisporus, Agaricus blazei, Lentinula edodes, Pleurotus ostreatus.

9. (canceled)

10: The method of claim 9 wherein said mushroom is enriched by pulsed UV irradiation.

11-16. (canceled)

17: The method of claim 10 wherein said Vitamin D content is increased to about 800% of the daily recommended value of Vitamin D and/or said mushroom's ergothioneine content remains unchanged after enrichment.

18-37. (canceled)

38: A nutritional product for improving athletic performance, muscle function and reducing injuries in mammals comprising a source of ergothioneine and/or a source of Vitamin D.

39: The nutritional product of claim 38 further comprising an antioxidant comprising N-acetyl cysteine.

40. (canceled)

41: The nutritional product of claim 38 further comprising n-acetyl cysteine (NAC), glutathione and/or turmeric.

42: The nutritional product of claim 38 wherein said source of Vitamin D is a UV irradiated, filamentous fungi, tissue, substrate or component thereof with increased levels of Vitamin D, and wherein said source of ergothioneine is a whole food or Cyanobacteria source.

43: The nutritional product of claim 38 wherein said source of ergothioneine comprises one of more of the cyanobacteria Spirulina, a cereal crop, or a naturally enhanced, filamentous fungi, tissue, substrate, spent substrate or component thereof.

44-47. (canceled)

48: A method for mobilizing iron in a mammal comprising:
administering to said mammal a source of Ergothioneine and/or Vitamin D;
wherein upon administration of said source of Ergothioneine and/or Vitamin D, iron is mobilized within functional pools or between functional pools in said mammal when compared to an mammal in the absence of such treatment; and
wherein said source of Ergothioneine and/or Vitamin D comprises a whole food or bacteria source.

49: The method of claim 48 wherein said mobilized iron within various functional pools or between functional pools comprises one or more of an increase in blood iron levels, an increase in total percentage iron saturation, and increase in oxygen carrying capacity.

50-51. (canceled)

52: The method of claim 48 wherein said source of Ergothioneine and/or Vitamin D comprises a whole food or bacteria source.

53: The method of claim 48 wherein said source of Ergothioneine and/or Vitamin D comprises a naturally enhanced, filamentous fungi, tissue, substrate, spent substrate or component thereof, with increased levels of Vitamin D.

54: The method of claim 53 wherein said filamentous fungi is a mushroom species enriched by pulsed UV irradiation and said Vitamin D content is increased to about 800% of the daily recommended value of Vitamin D and/or said mushroom's ergothioneine content remains unchanged after enrichment.

55: The method of claim 48 wherein said filamentous fungi is a mushroom species selected from the group consisting of: Pleurotus eryngii, Agaricus bisporus, Agaricus blazei, Lentinula edodes, Pleurotus ostreatus.

56: The method of claim 48 wherein said Vitamin D is Vitamin D₂.
